(19) United States
(12) Patent Application Publication
(10) Pub. No.: US 2021/0063330 A1
(43) Pub. Date: Mar. 4, 2021

(54) METHOD AND SYSTEM FOR DETERMINING THE CONCENTRATION OF CHEMICAL SPECIES USING NMR

(71) Applicant: University of Canterbury, Christchurch (NZ)

(72) Inventors: Yevgen MATVIYCHUK, Christchurch (NZ); Daniel Joseph HOLLAND, Christchurch (NZ)

(21) Appl. No.: 16/635,922

(22) PCT Filed: Aug. 10, 2018
(86) PCT No.: PCT/NZ2018/050111

§ 371 Date: Jan. 31, 2020

(51) Int. Cl. G01N 24/08; G01R 33/46; G01N 33/14; G16C 10/00

(52) U.S. Cl. CPC G01N 24/08; G01R 33/4625; G16C 10/00; G01N 33/146; G01N 33/143

(57) ABSTRACT

Method for determining the concentrations of constituent chemical species in a mixture, including the steps of: using nuclear magnetic resonance spectroscopy, acquiring an NMR measurement for a sample of the mixture; and for each of the constituent chemical species, retrieving a reference model representative of the NMR FID signal or frequency domain spectra from a database. Each model has a number of parameters, and for at least one of the constituent chemical species, the reference model is a quantum mechanical model. The method further includes, using a computer, generating a model signal for the mixture and adjusting some or all of the model parameters to fit the model signal to the measured data; and based on the fitted model signal, calculating and displaying the concentrations of the constituent species in the sample.

### FIELD OF THE INVENTION

This invention relates to a method and system to determine the concentration of constituent chemical species in a sample using nuclear magnetic resonance (NMR) spectroscopy, in particular, using medium-field NMR.

### BACKGROUND

Nuclear magnetic resonance (NMR) spectroscopy is a well-known non-destructive technique for mixture analysis. The technique involves applying an external magnetic field to a sample and causing excitation of the nuclei in the sample using radio waves. The resulting signals generated as the nuclei return to a resting state are detected using radio receivers, and commonly are converted to a spectral presentation using Fourier transform techniques.

Different chemical species each produce characteristic spectra with distinct peak patterns owing to differences in magnetic shielding and the resulting chemical shifts. The intensities of these peaks are proportional to the amount of related nuclei in the sample, therefore, the spectra can be used to determine the mole fraction of a chemical specie in a mixture.

Various techniques exist for estimating the relative quantities of constituent chemical species in a mixture from an NMR spectra. Most commonly, spectra are analysed using peak integration techniques. Peak integration is suitable for data sets in which separate non-overlapping peaks can be identified for each constituent species. However, even in these instances, peak integration techniques require specialist expertise and extensive manual processing particularly in the form of data pre-processing for phase and baseline correction. Some specialised techniques exist to integrate spectra with overlapping peaks, but these also require a high level of expertise and the methods still struggle with spectra that include regions with numerous adjacent peaks (such as the spectra of FIG. 2(i)).

In practice, obtaining spectra with lower numbers of overlapping peaks (as most suitable for peak integration) relies on the use of an instrument with high spectral resolution, and/or by restricting the samples to those of low complexity. High-resolution instruments, with high strength, homogenous magnetic fields, are expensive and large.

They typically require a dedicated space to house the instrument, making them largely the domain of universities and research institutes.

Recent developments in compact or ‘benchtop’ NMR instrumentation utilising non-cryogenic magnets have made NMR readily available for use in industrial applications in diverse fields. However, even though state-of-the art benchtop spectrometers now can deliver field homogeneity and functionality of more expensive superconducting magnets, their usefulness is limited by the low spectral resolution of the output.

In proton NMR, the most common mode of quantitative experiment due to its high signal to noise ratio and fast acquisition, peak splitting of coupled spins and overlap of peaks is common, even in molecules of only moderate complexity, e.g. monosaccharides. At the lower field strengths of benchtop instruments, chemical shift differences approach values of coupling constants; this introduces broad baselines and causes the shape of the peaks to completely change as groups of transition peaks split and multiple resonances overlap. FIGS. 1(i) and 1(ii) illustrate the difference in NMR spectra of propanol acquired at different field strengths; severe coupling distortions are apparent in the results from the benchtop instrument (FIG. 2(ii)). Consequently, peak integration becomes inaccurate for even moderately complex chemical mixtures using benchtop-type spectrometers.

It is known to model NMR spectra using parametric models. Such modelling approaches advantageously can be automated so to not require the same high level of expertise of the end user as peak integration, to analyse NMR spectra. The models regard a spectrum as a collection of Lorentzian peaks of different intensities, widths, and chemical shifts, further affected by additive Gaussian noise during acquisition. However, in practice, distortions seen in experimental data often violate the model assumptions. For example, insufficient homogeneity of the external magnetic field breaks the symmetry of the peaks, and diffusion processes in the sample contribute to additional peak broadening and cause their shapes to further depart from the ideal Lorentzian curves. Any residual signal remaining after model fitting may introduce bias in the fit and/or raise the overall uncertainty of quantification.

Furthermore, at the magnetic field strength of benchtop instruments, multiple closely spaced resonances corresponding to separate quantum transitions can be observed making neighbouring peaks overlap with each other. The resulting spectrum can no longer be analysed as a physically-meaningful collection of simple Lorentzian peaks. These change in peak shapes with changes in magnetic field strength makes existing parametric models specific to a given magnetic field strength, such that a model developed for a high resolution instrument is not appropriate for analysing results from a ‘benchtop’ instrument. Additionally, since each peak must be specified separately, this modelling method quickly becomes unwieldy and slow for samples with high numbers of peaks where the parameter space rapidly increases, particularly if the model is to be adjusted for experimental factors such as pH.

Finally, instead of parametric modelling, using experimentally obtained spectra for pure component chemical species is a further possible approach for analysing NMR signals, with some appeal for large molecules with many overlapping peaks in their spectra. However this approach is limited in that, even if the spectra of all components are available, they are only applicable for analysing data at the same field strength under the same experimental conditions. They do not accommodate different instruments with different field strength or even slight changes in spectra due to variations of experimental conditions such as pH.

It is an object of at least preferred embodiments of the present invention to address one or more of the above mentioned shortcomings and/or to at least provide the public with a useful alternative.

In this specification where reference has been made to patent specifications, other external documents, or other sources of information, this is generally to provide a context for discussing features of the invention. Unless specifically stated otherwise, reference to such external documents or sources of information is not to be construed as an admission that such documents or such sources of information, in any jurisdiction, are prior art or form part of the common general knowledge in the art.

### SUMMARY OF THE INVENTION

In a first aspect, the present invention provides a method of determining the concentrations of constituent chemical species in a mixture. The method comprises:

using nuclear magnetic resonance spectroscopy, acquiring an NMR measurement for a sample of the mixture; for each of the constituent chemical species, retrieving a reference model representative of the NMR FID signal or frequency domain spectra from a database, each model having a number of parameters; using a computer, generating a model signal for the mixture and adjusting some or all of the model parameters to fit the model signal to the measured data; and based on the fitted model signal, calculating and displaying the concentrations of the constituent species in the sample. For at least one of the constituent chemical species, the reference model is a quantum mechanical model.

The method is a computer implemented method. In an embodiment, the chemical species in the mixture are known but their quantities are unknown. A user may input the chemical species in the mixture, for example via a user form or user interface on a computer.

In an embodiment, the acquired NMR measurement is obtained using a benchtop-type NMR spectrometer, for example an NMR spectrometer of a type having a non-super-conducting, permanent magnet. In some embodiments the acquired NMR measurement is obtained using an NMR spectrometer with an operating frequency of less than 140 MHz, optionally less than 100 MHz, less than 80 MHz, or even less than 50 MHz, for example, 43 MHz. Alternatively, the measurements may be obtained using a high-field strength NMR spectrometer, for example one having a super-conducting magnet.

In an embodiment, the method further comprises the step of hierarchically arranging the reference models for the chemical species. This may involve forming one or more groups containing multiple constituent species, where constituent species within the same group display similar responses to specific experimental conditions. This allows one or more model parameters to be assigned at the group level to reduce the overall number of parameters and improve computing time. This step may be manually specified by an operator, for example, via a user interface or user-form. Alternatively the step may be automated.

In an embodiment, at least one of the constituent chemical species reference models is specified in terms of the transition peaks with parameters found by diagonalization of the spin Hamiltonian. Alternatively or additionally, at least one of the constituent chemical species reference models may be a quantum mechanical model utilising temporal propagation. For example, reference models for species having fewer than 12 or 13 coupled spins are preferably specified in terms of the transition peaks with parameters found by diagonalization of the spin Hamiltonian, whereas reference models for species having more than 12 or 13 coupled spins preferably utilise temporal propagation.

In an embodiment, different types of models are used to generate the reference signals of different constituent species. The models may each be selected from: a base model of spectra peaks, a quantum mechanical model utilising diagonalization, a quantum mechanical model utilising temporal propagation, and experimental data. Experimental data may be specific to the field strength of the NMR instrument and may be used to generate reference signals for solvents.

In an embodiment, the reference signal for at least one of the chemical species in the mixture is independent of the NMR instrument's field strength, for example by means utilising a quantum mechanical model utilising diagonalization, a quantum mechanical model utilising temporal propagation.

In an embodiment, the mixture contains K chemical species, and the NMR signal (x) for the mixture is a superposition of the reference signals uₖ of the constituent chemical species, modelled according to:

$$x = {e}^{i {ϕ}_{0}} {\sum }_{k = 1}^{K} {c}_{k} {u}_{k} ( {θ}_{k} , τ ) ,$$

where: θₖ represents the model parameters, τ is the ring-down delay, φ₀is the global phase shift and cₖ are intensity estimators that are proportional to the concentration of the corresponding species k.

In an embodiment, the model parameters comprise one or more of: chemical shifts of the peaks, relaxation rates, peak intensities, and J-coupling constants.

In an embodiment, the acquired NMR measurement is obtained using single pulse ¹H NMR.

In an embodiment, the method further comprises the step of using marginal posterior distributions of the intensity estimators to analyse the uncertainties in their calculated values.

In an embodiment, an MCMC algorithm is used to sample the posterior distribution of the fitted model to estimate the uncertainty of the model parameters.

In an embodiment, along with the step of displaying the concentrations of the constituent species in the sample, the confidence or credible intervals relating to the concentrations are displayed.

In an embodiment, the confidence or credible intervals are calculated using a robust variance estimator taking into account the residual signal between the model and the NMR measurements.

In an embodiment, the step of fitting the model signal, or calculating the concentrations of the constituent species further comprises the step of performing line shape correction.

In an embodiment, the step of fitting the model signal, or calculating the concentrations of the constituent species comprises utilising a generalized least squares (GLS) estimator, the generalized least squares (GLS) estimator treating possible model misspecification as additional non-isotropic noise. In an embodiment, the variance of the noise may be assumed to be proportional to the absolute value of the derivative of the NMR spectra.

In one embodiment, the mixture comprises soluble carbohydrates such as sugars. For example, the chemical species in the mixture may comprise one or more of: isomers of glucose, fructose, lactose and sucrose. Citric acid and/or malic acid may also be present. In one embodiment, the mixture may be a fruit juice.

In other embodiments, the mixture may comprise one or more of alcohols, such as propanol, vitamins, such as thiamine and pyridoxine, caffeine, opioids, amines, acids, or other substances. Preferably the constituent chemical species have known reference values of chemical shifts and J-coupling constants to facilitate the generation of quantum mechanical reference signals. Where reference values are not available, they may be fitted as additional parameters of the model or obtained using high-resolution NMR measurements.

In a second aspect, the present invention provides a system for determining the concentrations of constituent chemical species in a mixture, comprising: a nuclear magnetic resonance (NMR) spectrometer for acquiring an NMR measurement of a sample of the mixture; a computer storage medium comprising a database of NMR FID signal or frequency domain spectra reference models for each constituent species, each model having a number of parameters; wherein for at least one of the constituent chemical species, the model is a quantum mechanical model; a model signal generator configured to generate a model signal for the mixture and adjust some or all of the model parameters to fit the model signal to the measured data, and thereby calculate the concentrations of each of the constituent species in the sample; and a user interface for receiving input commands from a user and for displaying the calculated concentrations of each of the constituent species.

In an embodiment, the computer storage medium is a non-transitory computer-readable storage medium. For example, the storage medium may comprise one of more of a persistent memory device such as magnetic and/or optical disks, ROM, and PROM, or volatile memory such as RAM. The storage medium may be provided by or readable by a laptop or PC. Alternatively, the storage medium may be remote to the NMR machine, for example provided on a remote server.

In an embodiment, the model generator comprises means for combining the reference models for each constituent species to generate the model signal.

In an embodiment, the NMR spectrometer is a benchtop-type spectrometer, for example, of a type having a permanent, non-super-conducting magnet. The NMR spectrometer may have an operating frequency of less than 140 MHz, optionally less than 100 MHz, less than 80 MHz, or even less than 50 MHz, for example, 43 MHz. Alternatively, the measurements may be obtained using a high-field strength NMR spectrometer, for example one having a super-conducting magnet.

In an embodiment, the system comprises means for hierarchically arranging the chemical species and their respective reference models. This may comprise forming one or more groups containing multiple constituent species, wherein constituent species within the same group display similar responses to specific experimental conditions; and assigning one or more model parameters at the group level to reduce the overall number of parameters. The system may comprise user input means such as a user interface to enable a user to at least partially specify the hierarchical structure.

In an embodiment, at least one of the constituent chemical species reference models in the database is specified in terms of the transition peaks with parameters found by diagonalization of the spin Hamiltonian. These reference models are preferred for species having fewer than 12 or 13 coupled spins

In an embodiment, at least one of the constituent chemical species reference models in the database is a quantum mechanical model utilising temporal propagation. These reference models are preferred for species having more than 12 or 13 coupled spins preferably utilise temporal propagation.

In an embodiment, the database comprises different types of reference models for different constituent species, the model types being selected from: a base model of spectra peaks, a quantum mechanical model utilising diagonalization, a quantum mechanical model utilising temporal propagation, and experimental data.

In an embodiment, at least one of the reference models in the database is independent of the field strength of the NMR spectrometer.

In an embodiment, the model signal generator combines the reference signals uₖ for a mixture containing K chemical species using superposition, and generates the NMR signal (x) for the mixture according to:

$$x = {e}^{i {ϕ}_{0}} {\sum }_{k = 1}^{K} {c}_{k} {u}_{k} ( {θ}_{k} , τ ) ,$$

where: θₖ represents the model parameters, τ is the ringdown delay, φ₀ is the global phase shift and cₖ are intensity estimators that are proportional to the concentration of the corresponding species k.

In an embodiment, the model parameters comprise one or more of: chemical shifts of the peaks, relaxation rates, peak intensities, and J-coupling constants.

In an embodiment, the system comprises an error estimating module, configured to calculate the confidence or credible intervals using a robust variance estimator taking into account the residual signal between the model and the NMR measurements. The model signal generator may comprise the error estimating module. In an embodiment, the model signal generator may be configured to perform line shape correction.

In an embodiment, the system comprises an error estimating module for utilising a generalized least squares (GLS) estimator to fit the model signal to the NMR measurements, the generalized least squares (GLS) estimator treating possible model misspecification as additional non-isotropic noise. In an embodiment, the variance of the noise may be assumed to be proportional to the derivative of the NMR spectra.

In a third aspect, the present invention provides a non-transient computer readable medium containing program instructions for causing a computer to:
upon receiving an NMR measurement for a sample of a mixture of a number of known constituent chemical species, retrieve a reference model representative of the NMR FID signal or frequency domain spectra for each specie from a database, each model having a number of parameters; at least one of the retrieved reference models being a quantum mechanical model.
generate a computer readable model signal for the mixture and adjust some or all of the model parameters to fit the model signal to the measured data;
based on the fitted model signal, calculate and display the concentrations of the constituent species in the sample.

In one embodiment, the database is provided on the non-transient computer readable medium.

The non-transient computer readable medium may further comprise instructions to carry out one or more of the method steps described above in relation to the first aspect.

This invention may also be said broadly to consist in the parts, elements and features referred to or indicated in the specification of the application, individually or collectively, and any or all combinations of any two or more said parts, elements or features. Where specific integers are mentioned herein which have known equivalents in the art to which this invention relates, such known equivalents are deemed to be incorporated herein as if individually described.

The method and system described herein are described as being for ‘determining’ the concentrations of constituent chemical species in a mixture. It will be apparent to one of skill in the art that the output values will always have a margin of error, like all measurements, and therefore are estimations of the actual concentrations.

The term ‘comprising’ as used in this specification and claims means ‘consisting at least in part of’. When interpreting statements in this specification and claims that include the term ‘comprising’, other features besides those prefaced by this term can also be present. Related terms such as ‘comprise’ and ‘comprised’ are to be interpreted in a similar manner.

It is intended that reference to a range of numbers disclosed herein (for example, 1 to 10) also incorporates reference to all rational numbers within that range and any range of rational numbers within that range (for example, 1 to 6, 1.5 to 5.5 and 3.1 to 10).

Therefore, all sub-ranges of all ranges expressly disclosed herein are hereby expressly disclosed.

As used herein the term ‘(s)’ following a noun means the plural and/or singular form of that noun. As used herein the term ‘and/or’ means ‘and’ or ‘or’, or where the context allows, both.

### BRIEF DESCRIPTION OF THE DRAWINGS

The present invention will now be described by way of example only and with reference to the accompanying drawings in which:

FIGS. 1(i) and 1(ii) are NMR spectra for propanol, where FIG. 1(i) shows spectra obtained using a 400 MHz instrument, and FIG. 1(ii) shows spectra obtained using a 43 MHz ‘benchtop’ instrument;

FIGS. 2(i) and 2(ii) are NMR spectra for an apple juice sample, where FIG. 2(i) shows spectra obtained using a 400 MHz instrument, and FIG. 2(ii) shows spectra obtained using a 43 MHz ‘benchtop’ instrument;

FIG. 3 is a flowchart illustrating the steps and inputs in embodiments of the method according to the present invention;

FIG. 4 is an alternative flowchart illustrating the steps and inputs in embodiments of the method according to the present invention;

FIG. 5 is a schematic showing an exemplary arrangement of constituent chemical species in a hypothetical mixture into a hierarchical structure, to achieve efficiencies in computing time;

FIGS. 6(i) to 6(iii) are plots showing the effect of lineshape imperfection on quantification results, where FIG. 6(i) shows a spectra for thiamine, FIG. 6(ii) is an enlarged portion of the thiamine spectra, showing measured data and a fitted model without lineshape correction, with the non-Gaussian residuals shown in the lower panel, and FIG. 6(iii) is an enlarged portion of the thiamine spectra, showing measured data and a fitted model using lineshape correction to reduce model misspecification, with the non-Gaussian residuals shown in the lower panel;

FIGS. 7(i) and 7(ii) show measured NMR spectra for glucose, along with the modelled spectra for the component isomers, where FIG. 7(i) shows spectra obtained and modelled for a 400 MHz instrument, and FIG. 7(ii) shows spectra obtained and modelled for a 43 MHz ‘benchtop’ instrument;

FIGS. 8(i) and 8(ii) show measured NMR spectra for fructose, along with the modelled spectra for the component isomers, where FIG. 8(i) shows spectra obtained and modelled for a 400 MHz instrument, and FIG. 8(ii) shows spectra obtained and modelled for a 43 MHz ‘benchtop’ instrument;

FIGS. 9(i) and 9(ii) show measured NMR spectra for sucrose, along with the modelled spectra for the component isomers, where FIG. 9(i) shows spectra obtained and modelled for a 400 MHz instrument, and FIG. 9(ii) shows spectra obtained and modelled for a 43 MHz ‘benchtop’ instrument;

FIGS. 10(i) and 10(ii) show separate quantum transition peaks for glucose, where FIG. 10(i) is at high field strength, and FIG. 10(ii) is at medium field strength;

FIG. 11 shows changes in spectra of 0.3M glucose samples with variations in pH, with the chemical shift scale is positioned to match the anomeric doublet at 5.07 ppm;

FIGS. 12(i) to (iii) show differences between peaks of the anomeric proton in modelled spectra of β-glucopyranose as a result of a chemical shift deviation by 0.01 ppm, where FIG. 12(i) is simulated for a spectrometer frequency of 400 MHz , FIG. 12(ii) is simulated for a spectrometer frequency of 43 MHz, and FIG. 12(iii) shows the estimation error as a function of deviation in chemical shifts between the model and the data;

FIG. 13 is a plot showing average deviation of intensity estimates due to peak overlap expected with the model-based quantification approach of FIG. 3 with varying instrument field strength;

FIG. 14 is an overlay of multiple NMR spectra for a glucose solution, showing changes in the spectra with time, to monitor the interconversion of a-glucopyranose into its β form using a benchtop NMR instrument;

FIG. 15 is a plot showing changes in the quantified mole fractions of glucose isomers from the measurements of FIG. 14, with time, with an exponential curve fitted;

FIG. 16 is an example of one possible hierarchical model organisation of a sugar mixture or fruit juice;

FIGS. 17(i) and 17(ii) show experimentally obtained spectra of a juice (yellow kiwi) along with fitted models for the constituent sugar and acid species, where FIG. 17(i) shows spectra obtained and modelled for a 400 MHz instrument, and FIG. 17(i) shows spectra obtained and modelled for a 43 MHz ‘benchtop’ instrument;

FIGS. 18(i) to (iii) show results of analyzing compositions of various natural fruit juices where FIG. 18(i) shows results obtained with a high-field spectrometer, FIG. 18(ii) shows results obtained with a benchtop spectrometer, and FIG. 18(iii) shows reference values for some juices from the Nutrient Database of the US Department of Agriculture, for comparison;

FIG. 19 is a schematic showing an exemplary system for performing the method of FIGS. 3 and 4;

FIG. 19 is a schematic showing an exemplary system for performing the method of FIGS. 3 and 4;

FIGS. 20 (i) and 20(ii) illustrate experimental results and component reference models for alcohol mixtures using a benchtop NMR spectrometer, where FIG. 20(i) is for a first mixture with more ethanol than propanol, and FIG. 20(ii) is for a second mixture with more propanol than ethanol; and

FIG. 21 illustrates experimental results and component reference models or a mixture of vitamins obtained using a high-field spectrometer operating at 400 MHz ¹H frequency, enlarged detail views show overlapping peaks for the constituent species.

### DETAILED DESCRIPTION OF A PREFERRED EMBODIMENT

Theory

An NMR signal for a mixture of K chemical species can be modelled as a parametric model x consisting of a superposition of corresponding signature signals uₖ, k=1, . . . ,K weighted by the amount of the k^{th} chemical species in the solution, cₖ:

$$x = {e}^{i {ϕ}_{0}} {\sum }_{k = 1}^{K} {c}_{k} {u}_{k} ( {θ}_{k} , τ ) , \qquad ( 1 )$$

The sets of model parameters θₖ determine the appearance of each reference signal;

they are related to the nature of particular compounds and may include, for example, chemical shifts of the peaks, their relative intensities and widths, as well as values of J-coupling constants. Additionally, the global phase shift φ₀ and the ringdown delay τ bear the meaning of the zero- and first-order phasing terms, respectively.

The weighting coefficients cₖ, herein intensity estimators, are directly proportional to the amount of the corresponding species k. Therefore, estimating the weighting coefficients is the main goal of model-based quantification.

The generalised model above may be defined in the time domain or in the frequency domain by using suitable reference signals uₖ specified in the desired domain. Equation 1 does not set any inherent requirements on the reference signals uₖ and their parameterization. In general, the resulting model x is a complex valued vector of length N; depending on the chosen domain, it may represent either a free induction decay (FID) signal or the resulting spectrum.

Multiple approaches are available for modelling uₖ for a given chemical species and will be described in due course. For example, it is known to model uₖ by fitting a number of separate Lorentzian peaks or using experimental data. However, as discussed in the background section, these approaches have some shortcomings and are limited in their application.

Alternatively, a quantum mechanical model can be used to define uₖ. Such models utilise the characteristic that, even though peaks in an NMR spectrum can move as a response to changing experimental conditions, the spectrum is necessarily determined by the underlying molecular structure. Specifically, in an NMR experiment, one observes transitions between certain quantum states of the studied spin system. The appearance of all P transition peaks can be found based on the spin Hamiltonian of the quantum mechanical (QM) system, and in general can be expressed as some non-explicit function:

{ωₚ, bₚ, αₚ}ₚ₌₁^{P}=f^{QM}(δ, J, r) (2)

This function takes into account shielding effects experienced by different nuclei, and as a result—their individual chemical shifts δ the set of mutual J-coupling constants J,and possibly a relaxation model with rates r that defines the resulting peak widths [1] [2] [3]. Here, bold symbols denote arrays of parameters, with each entry corresponding to a specific nucleus in δ and r, or pairs of nuclei in J. Typically, due to splitting of resonances of coupled nuclei, the number of peaks observed in the spectrum is much larger than the number of spin chemical shifts and J-coupling constants.

Adopting the QM model formulation therefore reduces the number of free parameters to fit for improved computational efficiencies. In addition, because a QM model does not make any assumptions about the experimental conditions but rather describes a molecule itself, the same specification of a chemical specie is suitable for modelling NMR experiments with any types of pulse sequences run at any field strength.

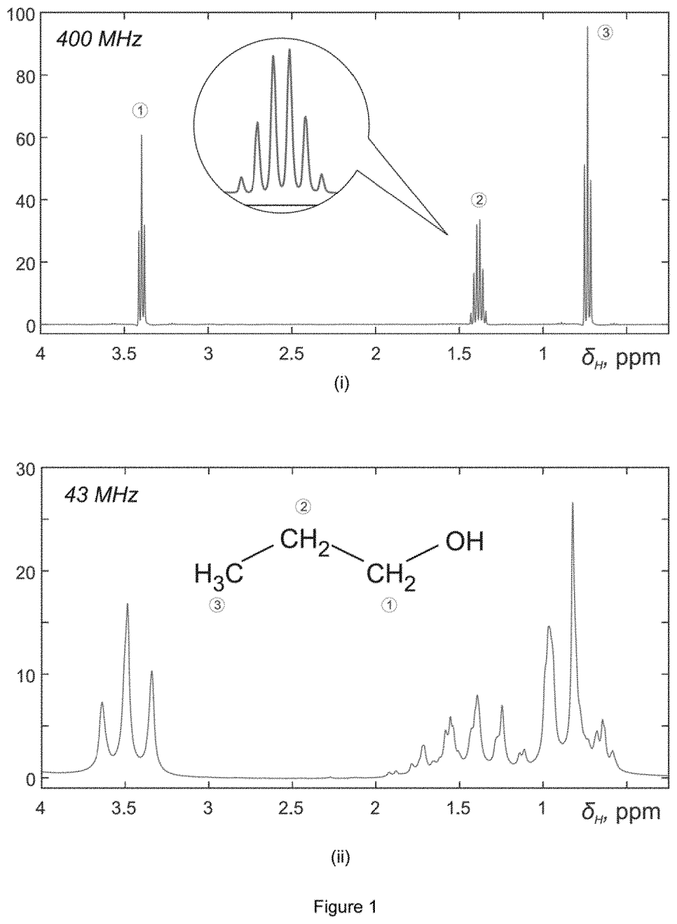

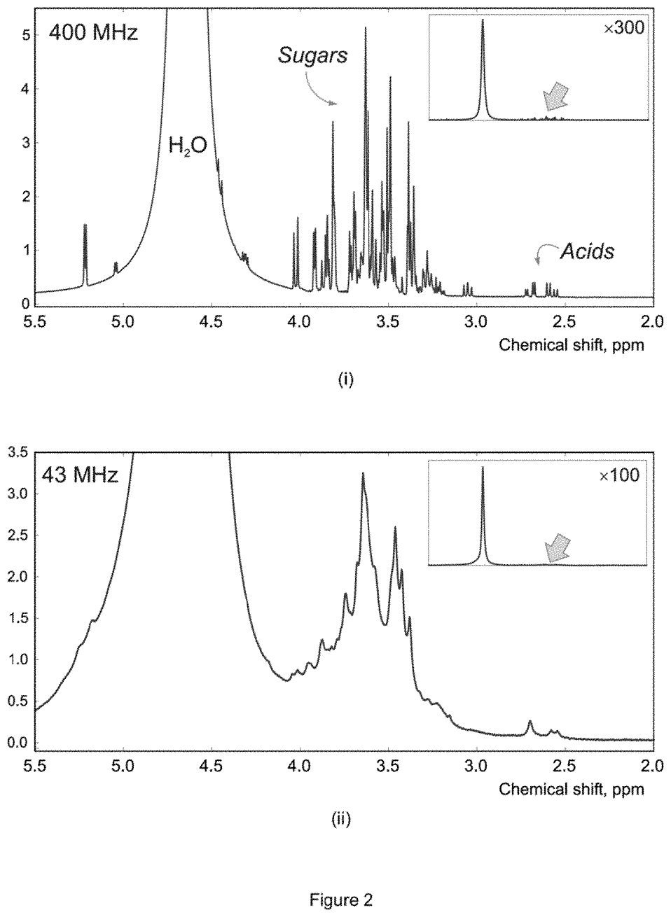

In the example of propanol shown in FIG. 1(i), the locations and intensities of the twelve peaks of propanol can be uniquely determined by only three chemical shifts and two coupling constants. The same set of five parameters also determine the more complicated spectrum obtained by the benchtop instrument and shown in FIG. 1(ii), which can be obtained simply by changing the field strength in the QM model f^{QM}. This field-invariance of the QM formulation advantageously enables the present method to process benchtop data.

The high-resolution structure of an NMR spectrum in terms of all transition peaks may be obtained using matrix formalism for representation of angular momentum operators and diagonalizing the Hamiltonian H of the spin system directly [4]. H is defined as a sum of the chemical shift (Zeeman) H_{Z} and coupling terms H_{J} as:

$$\begin{matrix}H = {H}_{Z} + {H}_{J} \\ = - 2 πγ {B}_{0} {\sum }_{s} [ 1 - {δ}_{s} ] {I}_{z} ( s ) + \\ 2 π {\sum }_{s , {s}^{′} > s} {J}_{s , {s}^{′}} [ {I}_{x} ( s ) {I}_{x} ( {s}^{′} ) + {I}_{y} ( s ) {I}_{y} ( {s}^{′} ) + {I}_{z} ( s ) {I}_{z} ( {s}^{′} ) ]\end{matrix} \qquad ( 3 )$$

Where: the indexes s and s′ run over all spins in the system and I_{X}; I_{Y}; I_{Z} denote their respective Cartesian spin operators; γ is a gyromagnetic ratio, and B₀ is the magnetic field strength in Tesla [5]. For a system of n spins, the above operators are defined as Kronecker tensor products of series of n [symbol] ^{2×2} matrices:

Iₓ(1)=σₓ⊗σ₀ . . . σ₀,

Iₓ(2)=σ₀⊗σₓ . . . σ₀,

Iₓ(s)=σ₀⊗σ₀ . . . σₓ . . . σ₀,

with similar equations for I_{y} and I_{z}, where σ₀ and σₓ, σ_{y} and σ_{z} are the identity operator and the three Pauli matrices, respectively:

$${σ}_{0} = [ \begin{matrix}1 & 0 \\ 0 & 1\end{matrix} ] , {σ}_{y} = [ \begin{matrix}0 & - i \\ i & 0\end{matrix} ] , \\ {σ}_{x} = [ \begin{matrix}0 & 1 \\ 1 & 0\end{matrix} ] , {σ}_{z} = [ \begin{matrix}1 & 0 \\ 0 & - i\end{matrix} ] .$$

That is, for each s=1, . . . ,n, in the sequence of Kronecker products σ₀⊗σ₀⊗ . . . ⊗σ₀, the s^{th} term is substituted with a certain Pauli matrix resulting in the corresponding 2ⁿ×2ⁿ operator, Iₓ(s), I_{y}(s), or I_{z}(s). Each eigenpair (h_{q}, λ_{q}) of the diagonalized Hamiltonian H=Σ_{q}λ_{q}h_{q}h_{q}^{T} corresponds to a certain quantum state from αα . . . α to ββ . . . β in the decreasing order of eigenvalues λ_{q} for q=1, . . . , 2n. If the transition from state q′ to state q″ is allowed, i.e. if an odd number of spins flip their state, the frequency of the corresponding resonance is found as ωₚ=2π(γB₀+λ_{q′}−λ_{q″}), and the intensity of the peak is bₚ=[h_{q′}^{T}h_{q″}]². Finally, a simple isotropic damping model for relaxation is used to assign widths to the resulting transition peaks, assuming that all nuclei in the same spin system relax with equal exponential rates leading to peaks of the same width. Optionally, for example, where high levels of computing power is available or where an isotropic damping model is less appropriate, other relaxation models may be used and the relaxation rates tailored for each transition peak.

### Modelling Individual Chemical Species

There are several distinct ways in which to specify a signature signal uₖ for a chemical specie. Table 1 outlines these various approaches—each approach takes a certain set of parameters as the input and results in a model for the NMR signal. Four approaches are detailed below, although other approaches may be possible. The choice of a suitable representation mainly depends on the nature of a particular chemical specie and the given application.

Referring to option 1 in Table 1, to obtain a modelled FID signal, a first option is to explicitly specify frequencies, intensities, and widths for all peaks in the spectrum. This approach is suited to relatively simple spectra with few peaks. Unless the spectrum contains only singlet peaks, such a model is field specific and not easily transferable to different field strengths. However, this approach can be useful to represent simple commonly occurring components, such as solvents.

Alternatively, a reference model can be specified in terms of the transition peaks with parameters found by diagonalization of the spin Hamiltonian (model 2), as described above. This approach offers the advantage of working with only a few higher-level parameters, such as chemical shifts and coupling constants attributed directly to the nuclei rather than to the observed peaks. Additionally, this modelling approach is not specific to the instrument field strength, so the same set of parameters can advantageously be used to easily find the transition peaks and generate a reference model for other operating frequencies. This method rapidly increases in complexity with increasing numbers of coupled spins due to the necessity of evaluating the exponential base model for each of the transition peaks, significantly slowing down computations. To improve computation time, it is possible to take advantage or the observation that many transition peaks have very small intensities, and many peaks are clustered together in just a few groups (observed as ‘usual’ peaks in the spectrum). Therefore, after diagonalization, closely located peaks can be combined and peaks with an intensity lower than a certain threshold can be discarded. It will be apparent that these simplifications will introduce some error, but depending on the thresholds selected, the error is usually negligible, and the process significantly improves computational time.

Even with such simplifications, for larger spin systems, it is more appropriate to use temporal propagation (model 3 in Table 1). Fast algorithms for temporal propagation have been developed recently to overcome the computational hurdle of Hamiltonian eigen decomposition [3] [6] [7]. Given values of chemical shifts, coupling constants, and a relaxation model, they directly compute the resulting FID and effectively realize a function u=f^{tQM} (δ, J, r). Such techniques are capable of handling much larger spin systems of over 40 spins or even 200 spins if state-space approximations are used [2]However these methods may result in suboptimal running times for smaller molecules (e.g. sugars), in which cases the direct diagonalization approach (model 2) is preferred.

For sufficiently large spin systems, polynomially-scaling propagation algorithms is the viable option to generate, possibly approximate, model signals. A potential downside is that since propagation techniques work by gradually evolving the resulting signal directly in the time domain, they effectively exclude information about separate transition peaks provided by the diagonalization methods, and this data can be valuable for certain applications.

Finally, a fourth option is to use an empirical signal of a pure component as a reference model. This option utilising experimental data is considered the least desirable approach but may be necessary for modelling very large molecules, whose complexity prevents quantum mechanical simulation or for specifying chemical species with unknown structure and for which spectra of the pure species are available. This method is expected to introduce the largest error into the final quantification results due to inevitable imperfections of phasing and baseline correction of the experimentally obtained reference signal as well as the presence of noise and possible lineshape distortions.

TABLE 1
Different methods of generating reference model signals [symbol] .

| Input parameters | Modeled FID |
|---|---|
| 1. Base model | |
| global shift \tilde{ω} and rate \tilde{α} | ũ = e [symbol] |
| peaks positions ω | u = ũ Σₚ bₚe^{[ω}^{ₚ}^{-α}^{ₚ}^{]}^{ₜ} |
| peaks intensities b | |
| relaxation rates α | |
| 2. QM model (diagoalization) | |
| global shift \tilde{ω} and rate \tilde{α} | ũ = e [symbol] |
| chemical shifts δ | ω, b, α = ƒ^{^{tQM}}(δ, J, r) |
| coupling constants J | u = ũ Σₚ bₚe^{[ω}^{ₚ}^{-α}^{ₚ}^{]}^{ₜ} |
| relaxation rates r | |
| 3. QM model (temporal propagation) | |
| global shift \tilde{ω} and rate \tilde{α} | ũ = e [symbol] |
| chemical shifts δ | u = ũ ƒ^{^{tQM}}(δ, J, r) |
| coupling constants J | |
| relaxation rates r | |
| 4. Experimental data | |
| global shift \tilde{ω} and rate \tilde{α} | ũ = e [symbol] |
| | u = ũ ƒ^{exp} (·) |
| 5. Group of species in fixed proportion | |
| global shift \tilde{ω} and rate \tilde{α} | ũ = e [symbol] |
| | u = ũ Σⱼcⱼuⱼ |

[symbol]

indicates data missing or illegible when filed

### Hierarchical Structure

Fitting a parametric model x to experimental data is the most computationally demanding step of the quantification procedure, which entails solving a multidimensional non-convex optimization problem. For example, to properly fit a QM model of an isomer of glucose, one needs to accurately estimate seven chemical shifts and possibly also seven J-coupling values. Other monosaccharides require finding similar numbers of parameters. Given a mixture of sugars, when multiple resonances overlap in a small spectral region, simultaneous fitting of all QM parameters becomes practically impossible if the spectral resolution is insufficient (e.g. with benchtop instruments).

Fortunately, observations indicate that, at least for some species, modelling deviations in individual peaks independently is often not necessary. However, their location as a group on the chemical shift scale may change (particularly when there is no unambiguous reference to set the 0 ppm mark). This suggests that when fitting signal models to datasets of certain chemical mixtures, to account for the move of the entire group of peaks, one may simply offset all chemical shifts in the QM model by the same amount \tilde{Ω}. Since such shift deviations are negligible comparing to the absolute resonance frequencies, they do not affect corresponding coupling patterns. Therefore, the computationally demanding QM simulations can be performed only once for some values in the middle of the range, and then the entire resulting spectrum can be shifted accordingly to fit the observations.

Furthermore, line-broadening caused by the loss of magnetic field homogeneity is likely to affect all peaks in the spectrum as well, and thus can be taken into account by controlling the widths of all peaks simultaneously with a single higher-level parameter.

If a reference model is generated using any of the methods discussed above, moving its peaks by \tilde{Ω} and applying Lorentzian broadening of rate \tilde{α} can be easily achieved by multiplying the modelled FID with a decaying harmonic signal, ũ=e^{[i\tilde{Ω}-\tilde{α}]t}. Optionally the broadening function may be a non-Lorentzian broadening, such as described later. By extension, one may even combine models of separate chemicals and adjust them simultaneously if desired (item 5 in Table 1). For example, if in a measured spectrum, all peaks of both anomers of glucose are shifted by the same amount with respect to their database values, this deviation is corrected by adjusting a single parameter \tilde{Ω} instead of fourteen chemical shifts. Such aggregation significantly reduces the effective dimension of the parameter space and facilitates model fitting. Nevertheless, after fitting these global parameters \tilde{Ω} and \tilde{α}, individual chemical shifts and J-couplings of each spin can be fine tuned if necessary.

The resulting reference signals uₖ defined in Table 1 can now be used to represent separate quantified constituents of the chemical mixture, either a single chemical, or a group of chemicals.

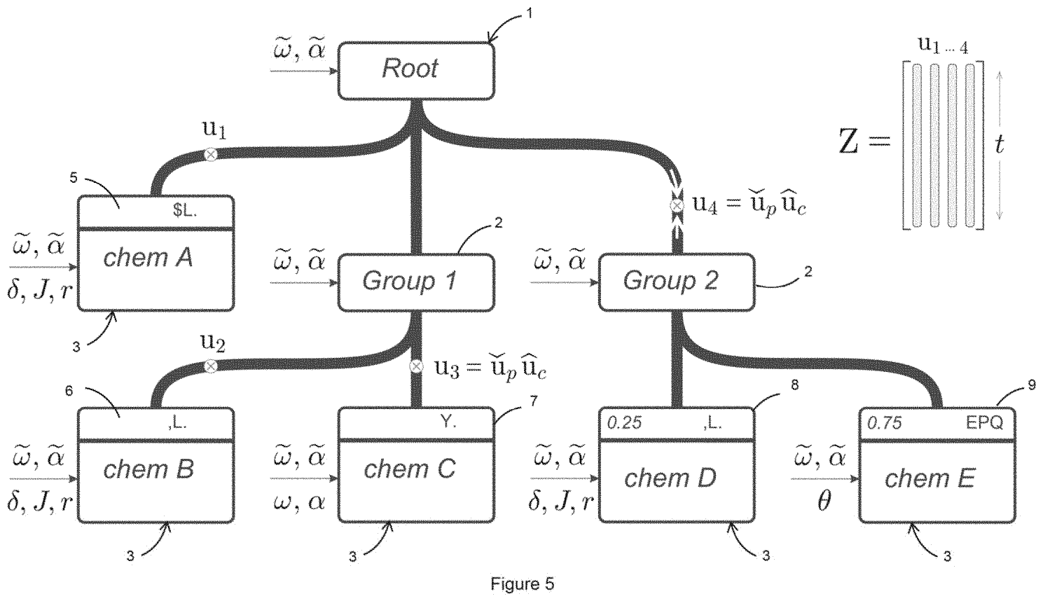

To allow for multilevel parameter organization, this idea of grouping chemicals can be performed in a hierarchical manner. FIG. 5 illustrates an exemplary hierarchical ‘tree’ structure for a hypothetical mixture of five chemical species, the structure comprises a number of nodes 1, 2, 3, which each represent either a single chemical specie or a group of chemicals. Each node is connected to a single higher-level parent node and/or has multiple lower-level child nodes. The terminal nodes 3 represent separate chemical species. Where these terminal nodes 3 have a parent node 2 that is not the root node 1, this signifies the inclusion of a chemical species into a sub-group (‘group 1’ or ‘group 2’ in FIG. 5). In general, parameters of any node in the tree, \tilde{Ω} and \tilde{α}, affect all its descendants equally.

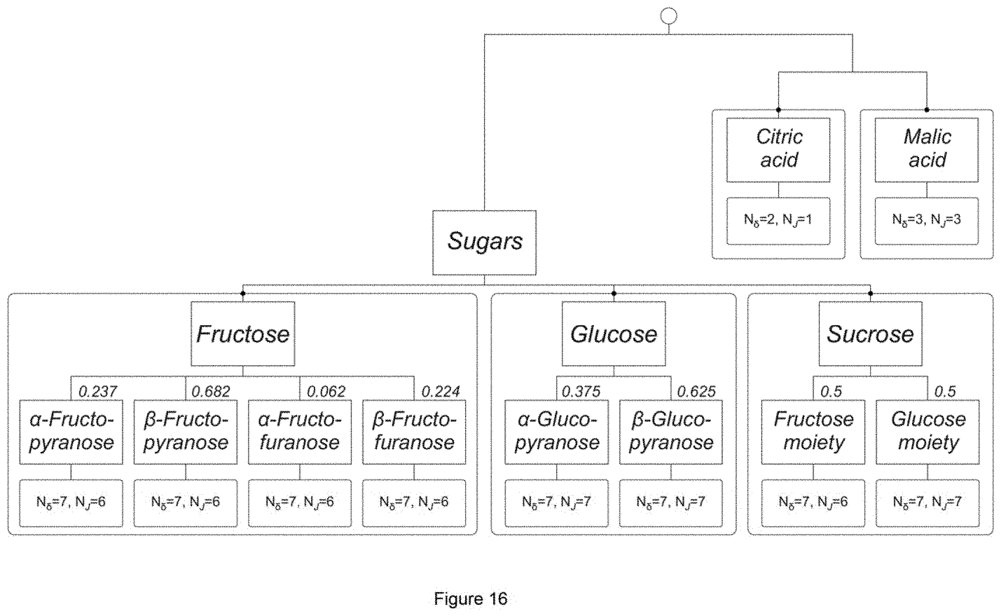

A further exemplary tree structure is shown in FIG. 16, where components in a fruit juice are grouped as sugars and acids, and, in turn, each reducing sugar is found in its several tautomeric forms. This model allows the signature models of all sugars, for example, to be controlled simultaneously and independently from the rest of the tree simply by adjusting parameters of the “Sugars” node. It will be apparent that this grouping of chemical species is not the only possible grouping—for example, alternatively furanose and pyranose isomers of all sugars may be assigned to different parent nodes if their models need to be altered in agreement with each other.

In the structure in FIG. 5, tree nodes are represented by functional blocks 5, 6, 7, 8, 9. Specifically, each block is defined by two internal signals, a signature u ū and a modifier ũ. The signature signal directly determines the functionality of the block. In the simplest case, an intermediate tree node that represents a group of chemical species, in addition to moving and broadening the peaks, may introduce some scaling to the resulting FID with ū=const. This functionality is useful for modelling mixture constituents whose mole fractions are known and fixed, e.g. the moieties of sucrose or glucose anomers at equilibrium.

As discussed above in relation to Table 1, depending on the chemical specie, different mathematical models may be used for different species. Therefore, different blocks may be parameterised differently depending on the model. For example, a signal from water is more conveniently described with the base model (BM, block 7) of single exponentially decaying sinusoid (time domain) or Lorentzian peak (frequency domain), while proper modelling of spectra of other molecules such as sugars can be performed by diagonalizing the spin system Hamiltonians (dQM, blocks 6 and 8). Moreover, for sufficiently large spin systems of more than 12-13 coupled nuclei, time-dimensional propagation of the quantum mechanical model may be feasible (tQM, block 5). Finally, experimentally obtained spectra can also be used as signature models (EXP, block 9). These different cases are represented by the different signal-generating blocks of four types as leaves in the tree model.

The second internal signal, a modifier ũ=e^{[i\tilde{Ω}-\tilde{α}]t}, is used solely to move and broaden the modelled peaks. Both signals are parametric functions of time. The specific sets of parameters θ depend on the type of a block and the represented chemical; in addition to the relative peak position \tilde{Ω} and decay rate \tilde{α} attributed to each block, they may also directly include absolute positions Ω, decay rates α, and intensities b of each peak separately or express them as functions of chemical shifts δ and J-coupling constants as indicated in Table 1 and FIG. 5.

Each block 2, 5, 6, 7, 8, 9 produces two output signals that are passed in one or both directions along the tree to the parent and/or child nodes, if any exist. The output in the upwards direction û aggregates signals of all species included in the corresponding subgroup. Given the internal signals ū and ũ, it is defined as û=ūũΣ_{c}û_{c} where the summation is over the output signals of all node's children. On the other hand, the output signal passed in the downwards direction encompasses shifting and broadening effects determined by all nodes located above. We define it as \check{u}=ūũ\check{u}ₚ, where \check{u}ₚ is the output of the node's parent. Now, a model signature signal for a group of chemical species represented by an intermediate node in the tree can be defined as:

$$u = {{u}^{⋁}}_{p} \hat{u} = {{u}^{⋁}}_{p} \overline{u} \tilde{u} {\sum }_{c} {\hat{u}}_{c} , \qquad ( 4 )$$

which represents a distinct quantified component of the mixture. Finally, by convention, we set \check{u}ₚ=1 for the root node and Σ_{c}û_{c}=1 for the leaves, and the above equation holds for all nodes in the tree.

For the exemplary mixture in FIG. 5, chemicals B and C are included as children of the same upper level node ‘Group 1’. This is because they are assumed to be affected in similar ways by changes in experimental conditions (e.g. pH), and thus the spectra peaks of B and C's signature signals are expected to move together. Similarly, chemicals D and E form another group with its own modification parameters \tilde{Ω} and \tilde{α}. The Root node combines all chemicals in the mixture; changing its parameters shifts and broadens the entire model spectrum and is useful in adjusting the reference ppm scale.

It may be desirable to quantify the chemicals A, B, and C separately. For this, the reference signals u₁, u₂, and u₃ of quantified mixture components are defined according to u=\check{u}ₚû=\check{u}=\check{u}ₚũũΣ_{c} û_{c} at the corresponding blocks in the tree. Furthermore, as an example, chemicals D and E may be known to be present in 1:3 ratio, but their total amount is unknown. Therefore, their individual intensities are weighted accordingly (using c=0.25 and c=0.75 respectively), and single reference signal to be used for quantification, u₄, is defined at the ‘Group 2’ node.

Initialising and Fitting the Model

The above parametric model of an NMR signal for the measured data y can be concisely formulated in a matrix form as:

y=Zce^{iφ}^{₀}+n (5)

where Z ∈ [symbol] ^{N×K} is the model matrix, whose columns contain ideal responses for each component/group that is, the reference signals, c ∈ [symbol] ^{K×2} is a vector of intensities, φ₀ is the phasing term, and n is a normally distributed random vector of noise.

Typically, good initial values of model parameters (chemical shifts and J-couplings) are known either as a result of previous experiments or can be found in numerous NMR databases. However, to accurately match the reference models with spectra measured at different experimental conditions, often the model parameters require adjustment. For example, chemical shifts of individual peaks may differ slightly from the values reported in databases as a response to varying pH, temperature, concentration or other factors beyond control of the experimentalist.

As for any model-based quantification method, the objective is to find the estimates of model parameters \hat{θ}₁, . . . , \hat{θ}_{K}, \hat{φ}₀, and \hat{τ} that best explain the measured NMR data y. In turn, this provides a straightforward way for estimating the associated signal intensities ĉₖ (equation 1) and the mole fractions

$${\hat{χ}}_{k} = \frac{{\hat{c}}_{k}}{{\sum }_{k} {\hat{c}}_{k}}$$

of chemical species. Assuming that the affecting noise n is isotropic Gaussian, the optimal model is obtained by minimizing the Euclidean norm of the residual, ∥y−x∥,

$${min}_{{\{ {θ}_{k} \}}_{k = 1}^{K} , τ , {ϕ}_{0} , c} ‖ y - {Zce}^{i {ϕ}_{0 }} ‖ \qquad ( 6 )$$

where, in compact matrix notation, Z ∈ [symbol] ^{N×K} is a model matrix with columns Z_{:,k}=uₖ(θₖ, τ) for k=1, . . . , K and c=[c₁, . . . , c_{K}]^{T} is a column vector of intensities. Z implicitly depends on all \hat{θ}_{K} and τ. Given a fixed model matrix z evaluated with some values of \hat{θ}₁, . . . , \hat{θ}_{K}, \hat{φ}₀, and \hat{τ}, this leads to the ordinary least squares problem with respect to the intensities ĉₖ. Following known derivations [8], we obtain a real-valued (ordinary least squares ‘OLS’) estimate for the vector of component intensities:

ĉ_{OLS}=Re(\hat{Z}^{H}\hat{Z})⁻¹ Re(\hat{Z}^{H} [symbol] ) (7)

where [·]^{H} denotes the conjugate transpose of a matrix and \hat{φ}₀ is an estimate of the global phase shift [9] [10]:

[symbol]

=½∠{[Z̅^{H}y]^{T}[Z̅^{H}Z̅]⁻¹[Z̅^{H}y]} (8)

where ∠[·] denotes the argument of a complex number. The sought mole fractions Xₖ are computed as ratios of the entries of ĉ.

These results assume that suitable values of the underlying model parameters {θₖ}ₖ₌₁^{K} and τ are known. In practice, they may need to be estimated as minimizers of the original model fitting problem in Equation (6). After substituting the found estimates ĉ and \hat{φ}₀ and rearranging its terms, this problem reduces to unconstrained maximization of the variable projection functional [symbol] (θ₁, . . . , θ_{K}, τ)=ĉ^{T}|\hat{Z}^{H}\hat{Z}|ĉ.

The derived model fitting solution can be viewed as a special case of a more general Bayesian framework [8] [11], which potentially allows us to incorporate prior information about the fitted parameters into the problem and estimate the uncertainty of the found results. Specifically, the above result can be derived as the maximum likelihood estimator of the component intensities under the assumption that the affecting noise n is circularly symmetric zero-mean complex Gaussian with variance σ². This assumption on the noise distribution is supported by the principle of maximum entropy and the Central Limit Theorem as the observed noise is likely to be contributed by a large number of independent additive sources (e.g. thermal noise). Furthermore, since the Gaussian distribution of the noise is preserved by the Fourier transform, the same optimality conditions hold for models defined in the time as well as frequency domains allowing us to use either Equation (7) or (8) to define the reference models and resulting in the same estimates of intensities in both cases.

With these assumptions, the variance of the estimate of intensity cₖ is found as the scaled diagonal term of the inverse matrix Re(\hat{Z}^{H}\hat{Z})⁻¹ [8]. The associated 95% confidence interval is specified as cₖ±2\tilde{c}ₖ for k-1, . . . K, where [symbol] is the corresponding standard deviation and can be found as:

\tilde{c}ₖ=σ\sqrt{[Re(\hat{Z}^{\hat{Z}})⁻¹]ₖₖ,} (9)

Where [.]ₖₖ denotes the k^{th} diagonal entry of a matrix in the brackets. This definition of uncertainty takes into account the influence of noise and also the peak overlap. Indeed, if spectra of all signals described by the model matrix Z do not overlap, their inner products—the off-diagonal terms in the matrix Z^{H}Z—are 0, leading to a diagonal inverse matrix. In this case, assuming that the model fit is perfect with respect to the parameters, the uncertainty of the estimates is determined solely by the variance of noise σ². However, spectral overlaps and non-zero inner products between different columns of Z raise the diagonal terms in Re(\hat{Z}^{H}\hat{Z})⁻¹ and increase the variance of the resulting estimates accordingly.

The covariance matrix associated with Equation (7) can be expressed as Σ_{c}_{_{OLS}}=σ²Be(\hat{Z}^{H}\hat{Z})⁻¹. The diagonal entries of Σ_{c}_{_{OLS}} describe the uncertainty in the estimates and can be used to define the confidence intervals. When the assumed generative models for mixture components perfectly match the experimental data and the assumptions about the distribution of noise are correct, the above estimator is the minimum variance unbiased estimator for the vector of intensities.

As for any model-based approach, the present method is limited in its capacity to account for unforeseen variability in experimental datasets not fully explained by the strict model assumptions. Most notably, deviations of the peaks' lineshapes from the perfect Lorenzian curves has been long known to pose a significant problem to model fitting methods. Such deviations can be caused by various factors, for example, diffusion processes in the sample, in which case Gaussian or Voigt functions have been found to be more faithful representations of the underlying peak lineshapes. On the other hand, inevitable inhomogeneity of the magnetic field across the measured sample volume leads to additional distortions that usually affect all peaks in the spectrum similarly. Finally, weak couplings between distant nuclei, while not easily observed directly, may cause broadening of certain peaks, in which case simple single-resonance models can no longer accurately represent the resulting composite lineshapes. These imperfections do not change the total signal intensity and thus, unless several peaks overlap, they typically remain unnoticed in peak integration. However, even small deviations from the ideal lineshape may have significant effects on the results of model-based quantification.

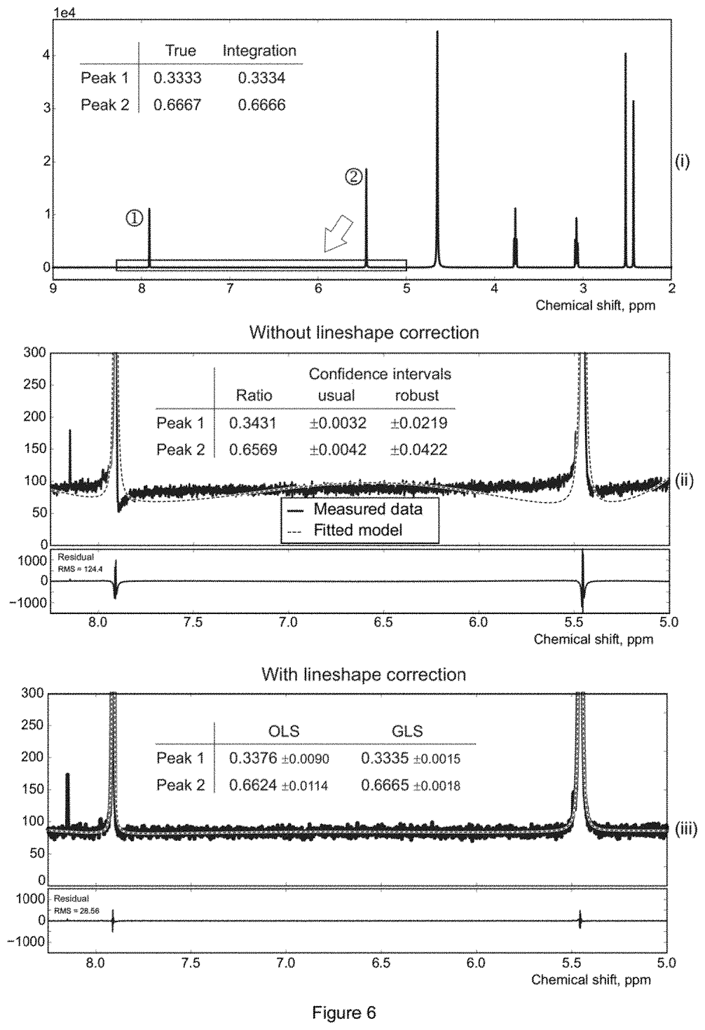

Referring to the example spectra in FIGS. 6(i) to 6(iii), the two peaks in the ¹H spectrum of thiamine shown in FIG. 6 correspond to uncoupled groups of one and two protons, and thus their intensities must be in a 1:2 ratio. Indeed, the ratios of peak integrals closely match the true values, however, fitting simple Lorentzian models to the peaks produces biased results.

Most importantly, the difference between the experimental spectrum and the estimated model, which ideally should be Gaussian noise, exhibits strong residual components. This violates the underlying assumption of the majority of model-based techniques that the error term n is circularly-symmetric Gaussian with zero-mean and fixed variance. As such, this model misspecification biases the estimated intensities and their variances. Assuming heteroscedasticity of the noise, a robust variance estimator can be constructed by taking into account the residual,

Σ_{c}_{_{OLS}}^{robust}=Re(\hat{Z}^{H}\hat{Z})⁻¹Re(\hat{Z}^{H}Ω\hat{Z})Re(\hat{Z}^{H}\hat{Z})⁻¹, (10)

where Ω is a diagonal matrix with entries equal to the squared values of the residual, y−Zĉ_{OLS}. However, this approach does not remove the bias in the intensities estimator ĉ_{OLS}.

The effects of lineshape imperfections can be partially alleviated by including a lineshape correction mechanism into the model as shown in the FIG. 6(iii). For example, modelling the FID envelope as a second-degree polynomial effectively allows us to account for the Gaussian lineshape distortions caused by diffusion. Unfortunately, even if all factors affecting the lineshape of each peak were known, explicitly including them into the generative signal model necessarily increases the number of free parameters needed to be estimated and is deemed impractical. Instead, we propose to approach the problem of defining the exact lineshapes with a degree of uncertainty and treat possible model misspecification as additional non-isotropic noise.

For this, noise in FIG. 6 is assumed normally distributed but with some general covariance matrix, Γ. Γ is unconstrained other than requiring it to be positive semidefinite. Therefore, the covariance matrix provides a convenient way to specify anisotropic or correlated errors, for example, to better model the residual in FIG. 6. In particular, when model fitting is performed in the frequency domain, errors caused by lineshape misspecification are likely to be larger where the signal changes faster, i.e. along the edges of peaks. In the example of FIG. 6 case, Γ is taken to be: Γ=σ²[(1−λ)I+λD], where D is a diagonal matrix with entries proportional to the absolute values of the derivative of the model spectrum normalized to ∥D∥₂=1. Other choices for the diagonal of D are possible, such as it being proportional to the values of the measured spectrum, the difference between the measured and modelled spectra, or a modelled spectrum convolved with any custom kernel. Furthermore, off-diagonal entries in Γ can be set to non-zero values to specify correlated errors. The adjustable scalar 0≤λ≤1 controls the relative amount of isotropic and anisotropic components of the noise and can be fitted with the rest of model parameters. With these definitions, the generalized least squares estimator of the intensities becomes:

ĉ_{GLS}=Re(\hat{Z}^{H}Γ⁻¹\hat{Z})⁻¹ Re(\hat{Z}^{H}Γ⁻¹ [symbol] ) (11)

and the covariance matrix Σ_{c}_{_{GLS}}=Re(\hat{Z}^{H}Γ⁻¹\hat{Z})⁻¹ specifies the uncertainty in the estimates. Similarly to the ordinary least squares case above, ĉ_{GLS} is the minimum variance unbiased estimator for the generalized problem. However, since the true Γ is unknown, this property cannot be guaranteed but produces tighter estimates of the intensities with notably reduced bias as shown in FIG. 6(iii).

Model Formulation in the Frequency Domain

The above described model is expressed in the time-domain, enabling simpler mathematical expression. However, it will be apparent to skilled persons that the model alternatively may be formulated in the frequency domain.

Assuming that an NMR signal is a superposition of Pₖ exponentially decaying harmonic components, an FID sampled at times t is defined as:

$${u}_{k}^{time} ( t , {θ}_{k} , τ ) = {\sum }_{p = 1}^{{P}_{k}} {b}_{p} {e}^{[ i {ω}_{p} - {α}_{p} ] ( t + τ )} . \qquad ( 12 )$$

The intensity coefficients bₚ in front of each component are proportional to the number of corresponding nuclei. The angular frequencies Ωₚ=2π(fₚ−fₒ) are usually defined with respect to some absolute) reference f₀ offset by the frequencies of chemical shifts of the peaks fₚ (in Hz). Finally, the decay rates αₚ, also expressed in Hz, are related to the full width at half maximum (FWHM) of each peak and the relaxation time T₂* through αₚ=πFWHM=1/T*_{w}. In this specific example, the triplets of parameters {bₚ, Ωₚ, αₚ}ₚ₌₁^{p-} constitute the sets θₖ for each mixture component. However, under the least squares criterion, when the noise is Gaussian, the equivalent frequency-domain formulation is possible. It can be easily obtained via the Fourier transform as is usually done in the traditional spectral analysis; alternatively, one may directly evaluate the Lorentzian function on the frequency grid f:

$${u}_{k}^{freq} ( ω , {θ}_{k} , τ ) = {\sum }_{p = 1}^{{P}_{k}} {b}_{p} \frac{{e}^{[ i {ω}_{p} - {α}_{p} ] τ}}{1 - {e}^{[ i ( {ω}_{p} - ω ) - {α}_{p} ] {Δ}_{t}}} , \qquad ( 13 )$$

where Δt is the sampling interval (dwell time).

As such, the model matrix z and the associated vectors x and y represent spectra instead of FID signals. This representation leads to the same formulation of the weighting coefficients, but is a preferable approach in cases when one is only interested in analysing only a portion of the spectrum because it is possible to select data only for the frequency range of interest, and thereby reduce the number of peaks/variables to fit.

Method

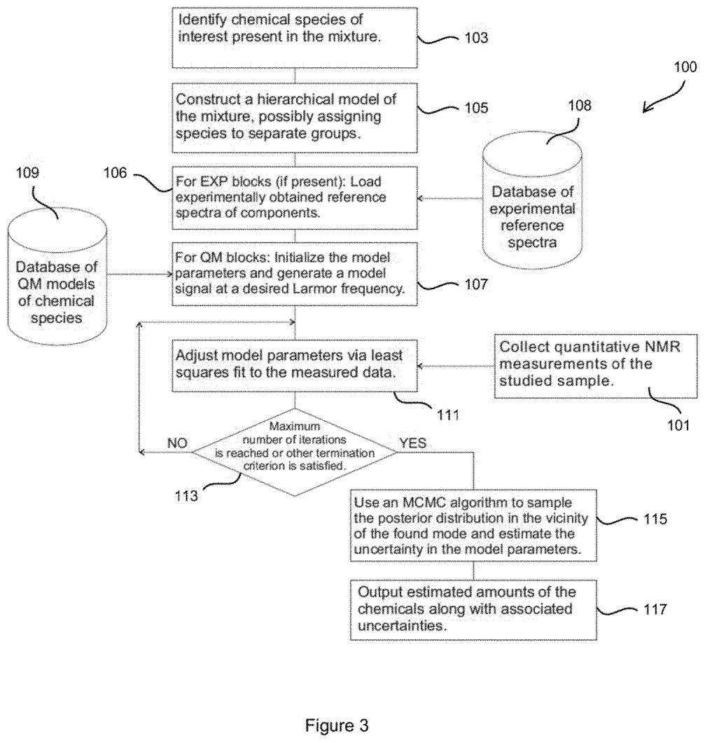

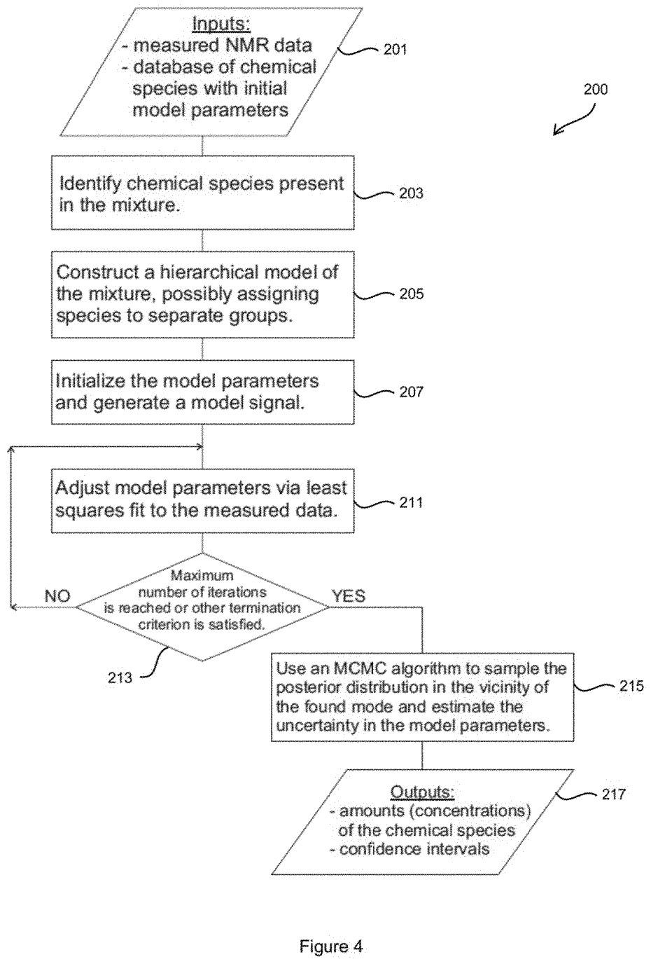

FIGS. 3 and 4 schematically illustrate an exemplary embodiment methods 100, 200 for determining the concentration of constituent chemical species in a sample using the principles outlined above. In an initial step 101, 201 a sample mixture is prepared for NMR spectroscopy according to the known process for the particular NMR instrument, and quantitative NMR measurements obtained for the mixture. The sample mixture is one that consists of known constituent species but in unknown quantities.

A user inputs information identifying the constituent chemical species, 106, 203 for example via a user interface on a laptop or PC, or via an input on the NMR instrument itself. The laptop or PC is preferably arranged to automatically or manually receive outputs from the NMR instrument.

The user constructs the hierarchical model, depending on the properties of the chemical species present in the sample, assigning each species to a group or sub-group of species in the sample for modelling purposes. Alternatively, this step may be automated and the chemical species analysed and grouped according to predetermined rules. That is, if there are two or more species having peaks that are expected to move together in response to changes in experimental conditions, they may be grouped together. A hierarchical model of the type illustrated in FIG. 5 is then constructed. In some mixtures, there may be no species that can be grouped together such that the hierarchical model consists of a root and only directly dependent terminal nodes representative of each species.

In a next step 106, 107, 207 models for each constituent species are pulled from a database 108, 109. As described above, the nature of the model depends on the specific chemical species, but where appropriate, the models are quantum mechanical models that are not specific to the field strength of the NMR instrument. Once model information for the constituent species is loaded, a model signal is generated at a desired Larmor frequency corresponding to the field strength of the NMR instrument (Ω₀=γ B₀, where B₀ is the field strength).

In a fitting step 111, 211, the computer takes the experimental measurements from the NMR sample and adjusts the model parameters to fit the model to the measured data using a least squares fit. Adjustment of parameters is carried out iteratively 113, 213 until an acceptable fit is reached, a maximum number of iterations is reached, or until the change in the values of the parameters between iterations is below a threshold value.

The amount/concentration of each chemical species is then calculated based on the intensity estimators from the resulting model.

Finally, the resulting model for the NMR signal is analysed at step 115, 215 to estimate the uncertainty of the intensity estimates due to noise and peak overlap, using one or more of the methods described above in relation to Equations 9 to 11.

Additionally, a Bayesian approach may be utilised. Methods for applying Bayesian statistics to modelling and sampling posterior distributions are known, for example, outlined in [8]. With the Bayesian approach, the joint distribution may be used to analyse the uncertainty in other model parameters such as chemical shifts and/or peak widths. When integrating out the parameters is difficult or not possible, the uncertainty can be estimated using a Markov chain Monte Carlo (MCMC) algorithm, sampling the Bayesian posterior distribution. MCMC sampling is only useful in the cases. This may be useful for characterizing the correctness of the models, for example.

The amount/concentration of each chemical specie is then output on the user interface along with the calculated uncertainty, which may be expressed as credible intervals at step 115, 215.

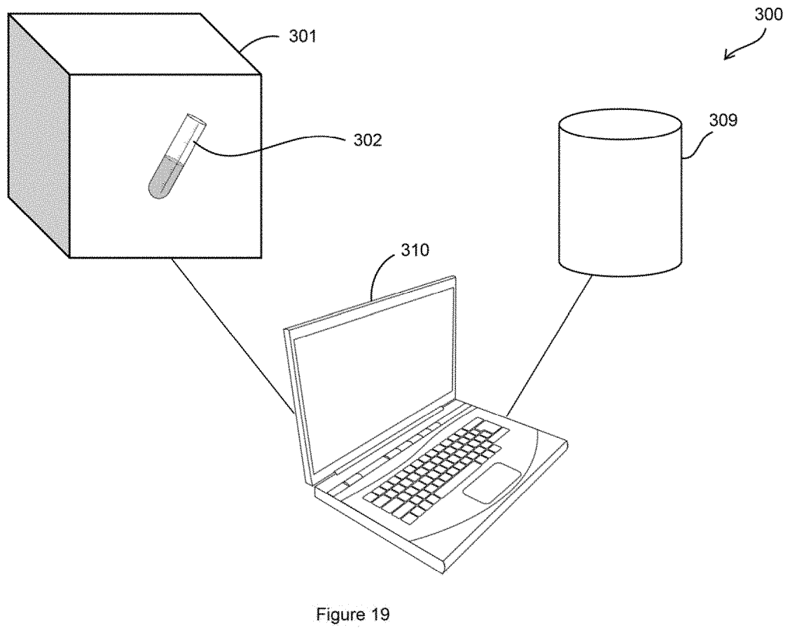

FIG. 19 schematically illustrates a system 300 for carrying out the method of FIGS. 3 and 4. The system comprises a nuclear magnetic resonance (NMR) spectrometer 301 for acquiring an NMR measurement of a sample 302 of the mixture. The database 309 is provided on a computer readable non transitory storage medium. A laptop or PC 310 is configured to receive outputs from the NMR spectrometer, inputs from a user, and also to access the database 309. The database 309 may be provided remotely, for example on a server and accessible over a network or internet connection. Alternatively, the database may be stored on internal non-transitory memory in the laptop or PC, or provided on portable/removable memory. The laptop or PC 310 comprises a processor for generating the model signal for the mixture, adjusting some or all of the model parameters to fit the model signal to the measured data, and calculating the concentrations of each of the constituent species in the sample. The instructions for the method are provided to the laptop, PC, or other computing device via a non-transient computer readable medium containing program instructions.

Applications

The present method enables a much wider application of benchtop-type NMR systems than is currently available. Therefore, this method has wide ranging applications where mixture analysis is required but large high-field NMR spectroscopy is impractical.

Food screening is one such potential application. Glucose, fructose, and sucrose—the three sugars most commonly occurring in fruit juices, wine, soft drinks, and other beverages—have different chemical properties and sweetness. Their relative concentrations (in addition to that of acids) largely determine the taste of a product. Therefore, knowledge of the exact composition of sugars in the mixture is important in the food industry, where it provides a means for process monitoring (e.g. fermentation), quality control, or even detection of adulteration (e.g. undisclosed sweetening of fruit juices or dilution of them with apple juice [12] [13]). High-field NMR spectroscopy has been shown to be particularly effective in these analyses, but these applications are often deemed too challenging for low-resolution benchtop instruments. The presence of multiple isomers of sugar species in relatively low concentrations, whose very complex overlapping spectra are further obstructed by the strong solvent peak are the main obstacles that prevent effective and accurate quantification.

Other food and beverage related applications include analysing alcohol content, testing for vitamins such as thiamine or pyridoxine, for example, or substances such as caffeine, or simple acids (e.g. malic, citric, maleic, lactic, etc.) and aminoacids.

The method also has application in forensic science, particularly in testing for certain opioids. Or in the chemical industry, for example to detect CO₂ absorption in amines, or the purity of chemical products.

In general, the method is suitable for ¹H NMR, for any molecule provided that reference values of chemical shifts and J-coupling constant are given or can be obtained from a high resolution dataset. Molecules containing (any number of) spin systems with no more than 12-13 coupled protons each can be modelled with the diagonalization approach, and temporal propagation techniques are used to model larger spin systems. The success of the quantification method for the analysis of a particular mixture critically depends on the number of mixture components, the field strength of the spectrometer, and as a result, the extent of peak overlap. This can be analysed numerically on a case by case basis by considering the properties of the model matrix Z^{H}Z.

For ¹³C NMR spectroscopy for this application where no coupling is observed, the present method still provides advantages. The limiting factor in ¹³C NMR is the level of noise in the dataset. In this regard, the robustness of the present model-based approach to high levels of noise is important for accurate quantification and the present method compares favourably with traditional peak integration.

Experimental Results

Analysis of Sugar Mixtures

The above described system and method is herein illustrated experimentally for samples containing controlled mixtures of sugars and samples of natural fruit juices. High-field

NMR spectroscopy has been proven an excellent technique for analysing compositions of various food samples as well as in numerous conformational studies of carbohydrates. However, most sugars have a highly complex ¹H NMR spectra, largely precluding the traditional peak integration analysis, especially of data observed on medium-field benchtop instruments. In addition, the water peak is located closely to the spectra of sugars, significantly distorting the baseline and complicating the analysis. Therefore, using the sugar mixtures described herein cannot be readily quantitatively analysed using medium-field benchtop NMR instruments and existing techniques.

In addition to the relatively high number of coupled protons (6-7 in most monosaccharides), reducing sugars such as glucose and fructose, exist in solutions in several tautomeric forms. For example, α- and β-glucopyranose, which under normal conditions account for approximately 37.5% and 62.5% respectively of glucose in aqueous solutions [14]. For fructose, four tautomers, namely α- and β-fructopyranose and α- and β-fructofuranose, can be observed in solution in considerable amounts (2.67%, 68.23%, 6.24%, 22.35% in tautomeric equilibrium at 20° C. [15]. (We disregard the intermediate open aldehyde form, which typically accounts for only 0.05% of fructose in solution). The tautomeric differences in the molecular structure affect magnetic shielding of even distant protons and thus alter their chemical shifts and J-coupling constants. As a result, these different isomers are distinguished by NMR spectroscopy and thus need to be modelled separately with their own reference models.

For the below described experiments, samples were measured using two NMR spectrometers: a medium-field Magritek Spinsolve benchtop system operating at a ¹H frequency of 43.6 MHz; and on a 400 MHz Agilent 400MR spectrometer equipped with a OneNMR probe. On the benchtop system, ¹H FID signals were acquired with 32768 points and dwell time of 200 μs using a one-pulse sequence with pulse angle 545 of 90°; datasets with variable numbers of scans were acquired with repetition time of up to 60 s. Likewise, on the high-field instrument, 16384 time points were measured with dwell time of 312.5 ps and pulse angle of 45°; a coaxial capillary with D₂O was inserted into the sample tube to establish the lock signal for the high-field spectrometer.

Development and Validation of Reference Models for Sugars

Using the quantum mechanical description of the spin systems as given in Table 1, reference signals are defined for each isomer. For the purpose of analysing ¹H NMR datasets, each isomer of the considered sugars is modelled as a spin system of 7 coupled protons. A molecule of sucrose is regarded as comprising two uncoupled moieties, glucose and fructose, present in equal amounts and model them with separate QM formulations. Therefore, 7 chemical shifts and 6-7 J coupling constants need to be estimated for each spin system to completely specify the model of the isomer. These parameters have been previously assigned using high-field NMR data and are reported in the literature [15] [16] [14]. However, differences in experimental conditions in these sources (for example the presence and amount of D₂O in solutions added to provide a lock signal for the spectrometer) may notably affect the reported chemical shifts and are difficult to account for consistently.

To avoid these inaccuracies and confirm the parameter values, for each or glucose, fructose, and sucrose, a spectra of ≈0.5M pure equilibrated sugar solutions in 100% deionized water is acquired on the ‘high field’ 400 MHz spectrometer. QM model signals are generated for each case using Equation 2, and their parameters are then fine-tuned. The values found for each studied isomer are given in Table 2. These values agree with the assignments reported elsewhere [17] [16] [18] [14] [15].

TABLE 2

| | H₁ | H₁ₐ | | H₂ | H₃ | H₄ | H₅ | H₆ₐ | H_{6b} |
|---|---|---|---|---|---|---|---|---|---|
| | ³J₁₋₂ | ³J_{1a-1b} | H_{1b} | ³J₂₋₃ | ³J₃₋₄ | ³J₄₋₅ | ³J₅₋₆ₐ | ³J_{5-6b} | ³J_{6a-6b} |
| α-GP | 5.230 | | | 3.531 | 3.709 | 3.408 | 3.824 | 3.840 | 3.764 |
| | 3.817 | | | 9.732 | 9.336 | 10.357 | 1.870 | 5.349 | −12.129 |
| β-GP | 4.641 | | | 3.239 | 3.483 | 3.401 | 3.459 | 3.896 | 3.722 |
| | 8.326 | | | 9.158 | 9.389 | 9.604 | 2.001 | 5.962 | −12.419 |
| α-FF | | 3.646 | 3.658 | | 4.105 | 3.993 | 4.054 | 3.817 | 3.695 |
| | | −12.090 | | | 4.220 | 7.244 | 3.043 | 5.508 | −11.900 |
| β-FF | | 3.590 | 3.551 | | 4.108 | 4.105 | 3.822 | 3.799 | 3.674 |
| | | −12.350 | | | 8.500 | 7.572 | 2.976 | 6.368 | −12.660 |
| α-FP | | 3.707 | 3.650 | | 4.033 | 3.941 | 3.863 | 3.857 | 3.707 |
| | | −11.680 | | | 6.729 | 2.954 | 1.574 | 2.550 | −12.790 |
| β-FP | | 3.708 | 3.560 | | 3.792 | 3.891 | 3.995 | 4.017 | 3.701 |
| | | −11.730 | | | 9.978 | 3.554 | 1.121 | 1.753 | −12.930 |
| Su-F | | 3.676 | 3.676 | | 4.211 | 4.044 | 3.884 | 3.828 | 3.809 |
| | | −12.500 | | | 8.751 | 8.469 | 2.723 | 6.952 | −12.310 |
| Su-G | 5.440 | | | 3.558 | 3.756 | 3.470 | 3.832 | 3.816 | 3.818 |
| | 3.350 | | | 9.997 | 9.076 | 10.130 | 2.244 | 4.329 | −14.440 |

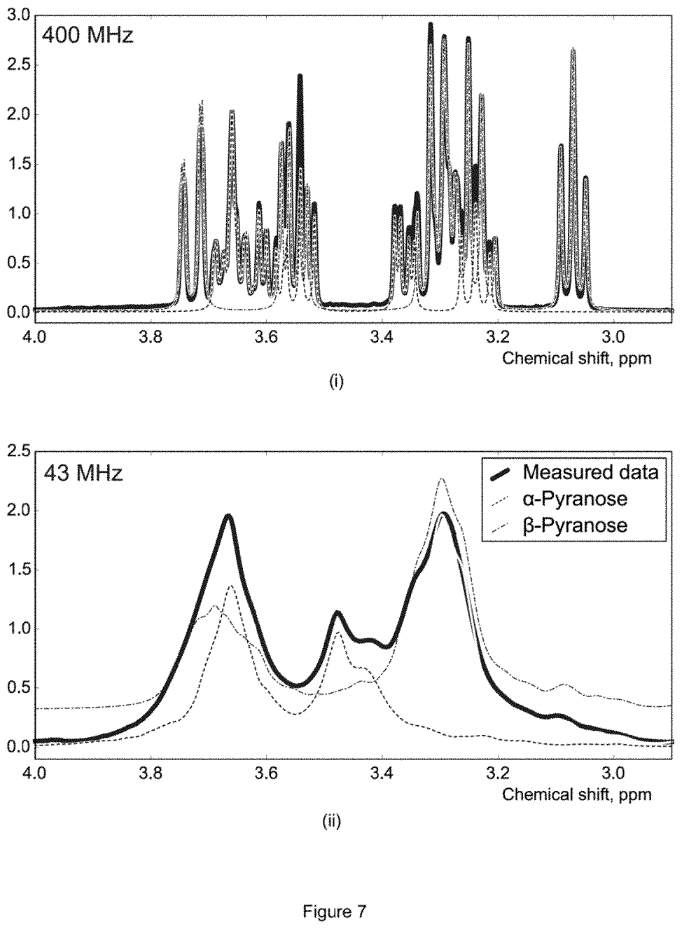

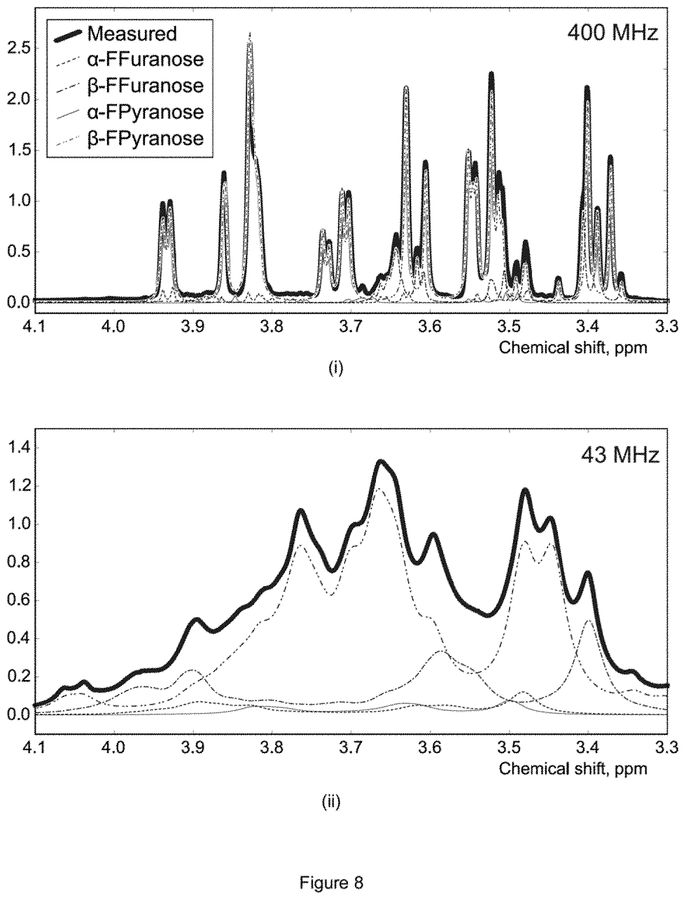

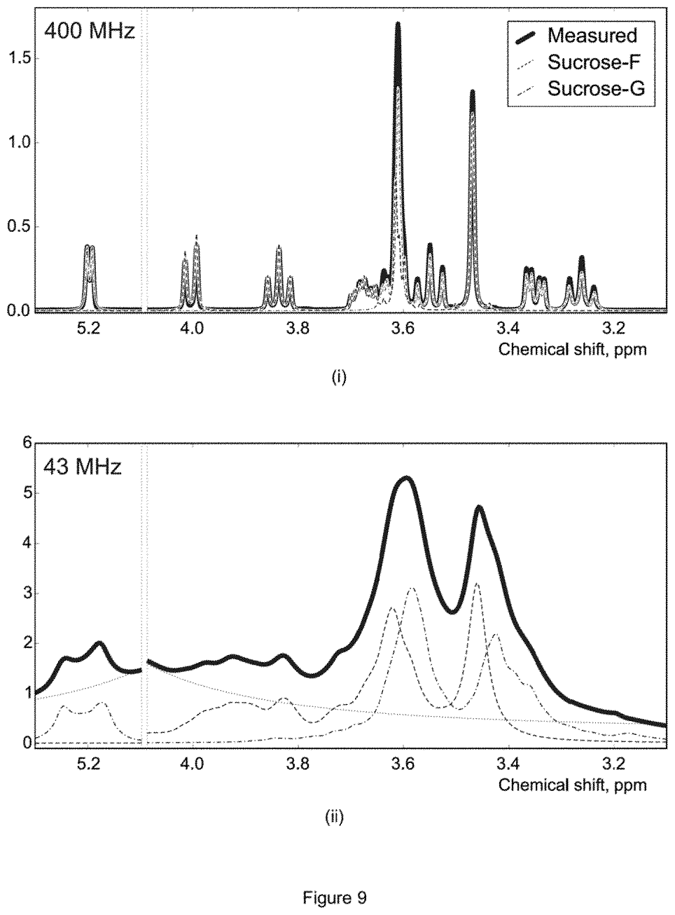

FIG. 7(i) shows a good match between the generated models of glucose isomers and the experimental spectra of an equilibrated solution of pure glucose measured on the high-field spectrometer. The root-mean square (rms) of the residual is 0.067, about 30 times lower than the average height of the glucose peaks 2.0). Similarly, FIGS. 8(i) and 9(i) compare the model with the experimental spectra for the four most abundant isomers of fructose (FIG. 8(i)) and the two components of sucrose (FIG. 9(i)), also showing a good match.

To validate the models for lower-field data, ¹H spectra of the same three samples of pure sugars were obtained using the benchtop NMR instrument. Using the proton chemical shifts and J-coupling constants measured using the high field instruments, signal models were generated corresponding to the 43 MHz operating frequency, and fitted to the data by adjusting only the position of the entire group of the peaks and their relaxation rate. FIGS. 7(ii), 8(ii), and 9(ii) illustrate the match between observed benchtop data and the fitted models for the three samples respectively. It can be observed that resonances attributed to different protons can no longer be separated in these lower field spectra.

Instead, the spectra is an ensemble of closely spaced and highly overlapping peaks corresponding to different quantum transitions of these large spin systems.

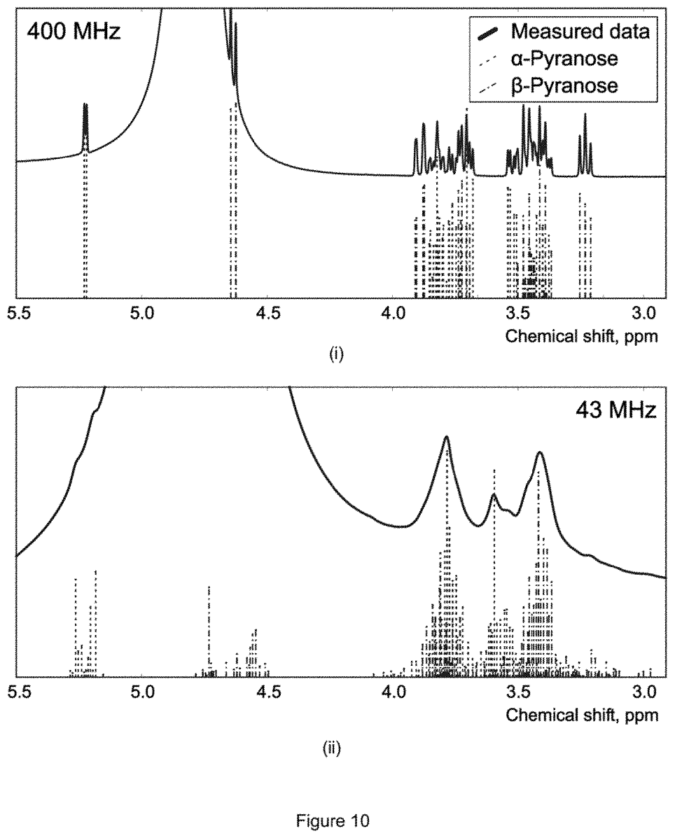

NMR spectra obtained using Benchtop instruments typically have lower signal to noise ratios than high field data and are considered to have poorer line shape. However, the line shape imperfections seen in the benchtop NMR data in FIGS. 7(ii), 8(ii), and 9(ii) are not due to these effects but rather arise from the different quantum transitions of these large spin systems. To illustrate this, FIG. 10(ii) displays the modelled resonances for the isomers of glucose at medium field. Clearly, asymmetry of peaks and small b umps near the baseline observed in the experimental spectra arise from superposition of the transition peaks. They are well explained and fitted by the quantum mechanical models used and transferred to the medium field. The models achieve an rms of the residual of only 0.036—two orders of magnitude lower than the dynamic range of the glucose peaks (≈2.0) and four orders of magnitude lower than the height of the neighbouring water peak (262.5).

In a next step, the models obtained for each sugar isomer are used to quantify the relative concentrations in solution, both as measured with the high and medium field strength spectrometers. Table 3 compares the results with their reference values found in the literature ([14] for glucose and [15] for fructose) or the ground truth (for sucrose). The error for the medium-field values does not exceed 1% in relative concentrations of isomers and is less than 0.5% with high-field data. The reported 95% credible intervals are computed according to Equation (9) , where G is estimated based on the difference between the measured data and the fitted model. Therefore, in addition to the effect of noise, the estimated uncertainties include the effect of slight model misfit due to possible errors in model parameters. Notably, the estimates obtained at both field strengths have comparable confidence bounds.

TABLE 3
Relative equilibrium concentrations of sugar isomers in aqueous
solutions found using data from 400 MHz spectrometer and
43 MHz benchtop instrument; the estimates are reported along with
their 95% credible intervals.

| | Reference | 400 MHz | | 43 MHz | |
|---|---|---|---|---|---|
| α-GP | 0.375 | 0.373 | ±0.007 | 0.379 | ±0.006 |
| β-GP | 0.625 | 0.627 | ±0.009 | 0.621 | ±0.007 |
| α-FF | 0.062 | 0.051 | ±0.005 | 0.053 | ±0.008 |
| β-FF | 0.224 | 0.228 | ±0.007 | 0.241 | ±0.010 |
| α-FP | 0.027 | 0.023 | ±0.007 | 0.019 | ±0.009 |
| β-FP | 0.682 | 0.698 | ±0.011 | 0.688 | ±0.016 |
| Su-F | 0.5 | 0.498 | ±0.006 | 0.502 | ±0.005 |
| Su-G | 0.5 | 0.502 | ±0.006 | 0.498 | ±0.005 |

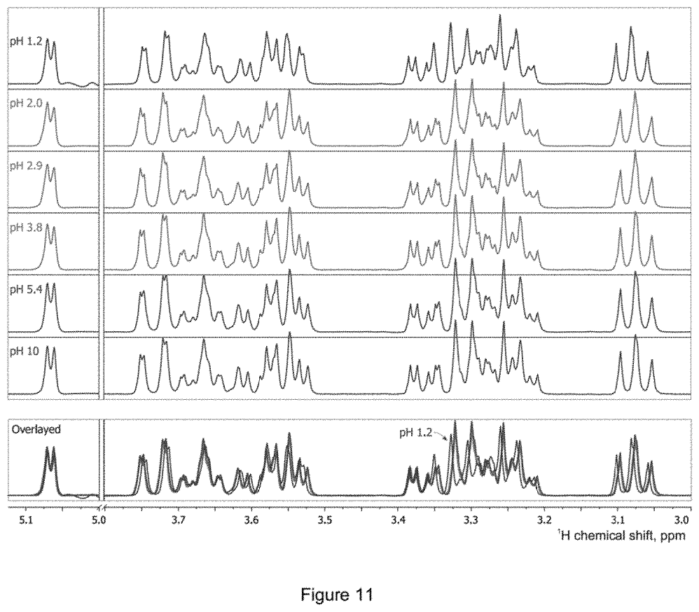

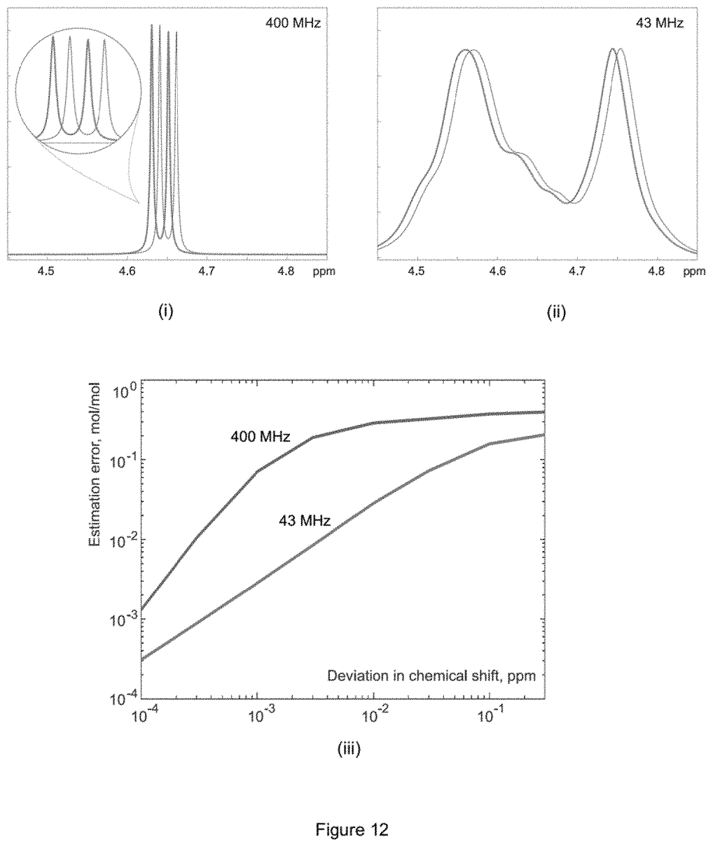

The present model-based quantification of sugar mixtures with benchtop NMR, relies on the field invariance property of QM models—assuming that chemical shifts and J-couplings obtained using well resolved high-field data remain unchanged in different samples. FIG. 11 shows spectra of 0.3M glucose samples with varying pH from 1.2 to 10. The chemical shift scale is positioned to match the anomeric doublet at 5.07 ppm across the samples. Note that deviations in the relative positions of individual peaks occur only at the extreme pH of 1.2. In the pH range of most fruit juices (2.2 . . . 4.6), positions of the glucose peaks can be considered fixed. Similar results are observed for fructose and sucrose. In addition, experiments with multiple samples of natural fruit juices described further below, whose pH range from 2.2 for lime juice to 4.6 for mango juice, corroborate the assumption that peaks of the studied sugars do not shift noticeably with respect to each other, and their spectra are effectively fixed. However, the present method allows for changes in widths and positions of all peaks simultaneously when fitting models for each particular experiment.

To estimate possible errors due to model misfit, we run the following numerical simulation. Using the model values listed in Table 2, we generate synthetic signals for the mixture of glucose isomers taken in equal amounts at 400 and 43 MHz ¹H spectrometer frequencies. We then apply the present quantification method, but, to simulate the uncertainty in the experimental parameters, we randomly deviate all chemical shifts in the model with respect to the “true” values used to generate the data. Interestingly, the same deviation in chemical shift leads to much higher discrepancy between the model and the data at higher operating frequency than at 43 MHz as shown in 12 (i) to (iii). This demonstrates that, all other conditions being equal, model-based quantification of benchtop data is more robust to inaccuracies in chemical shifts of the peaks (e.g. as a result of changing pH) than its higher resolution 400 MHz counterpart. Model misfit errors such as these will give a lower limit on the estimated error that cannot be removed even with improvement in the signal to noise ratio of the measured data, but can be mitigated by additional fitting of underlying model parameters where possible.

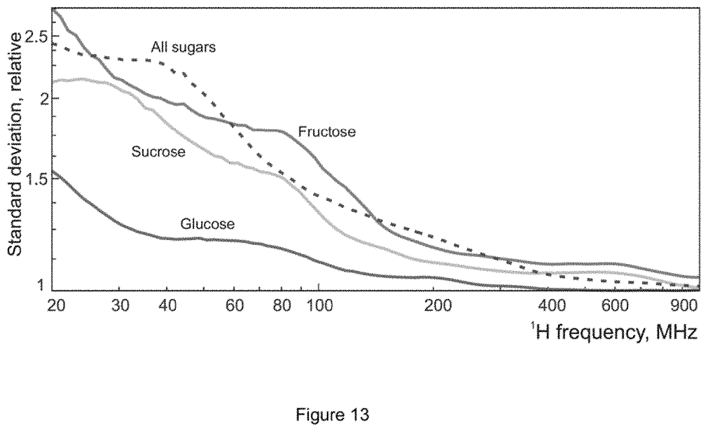

In contrast, the uncertainty in the estimates inevitably increases at lower field strength due to significantly reduced spectral resolution. The effects of peak overlap on the estimation accuracy can be conveniently quantified with Equation (9) assuming fixed variance of noise σ²=1. Using datasets simulated at various field strengths, FIG. 13, shows average estimated values of deviations of component intensity estimators ,

$$\tilde{c} = \frac{1}{K} {Σ}_{k} {\tilde{c}}_{k} ,$$

for mixtures of isomers of glucose and fructose and also for the two components of sucrose modelled separately. The solid lines correspond to estimating relative concentrations of isomers of glucose and fructose as well as the two components of sucrose; the dashed line is for the case of analyzing mixtures of the three sugars (while holding ratios of their isomers fixed in the models. As expected, the analysis of mixtures containing four fructose isomers is a more challenging problem with higher associated uncertainty than the quantification of binary mixtures of glucose isomers or sucrose components. In the latter cases, the non-overlapping peaks of anomeric protons in spectra of glucose located further downfield from the main group of peaks enable more accurate quantification. Importantly, these simulations assume that the true chemical shifts and J-couplings are known. With experimental data, their estimation inevitably contributes to additional errors. Since high-field models are more sensitive to the accuracy of parameters, their resulting uncertainty is comparable to that of the values obtained at 43 MHz, as we observe in Table 3.

Monitoring Interconversion of Glucose in H₂O

The ability of NMR spectroscopy to distinguish different structural isomers makes it an important tool for studying kinetics of interconversion reactions of reducing sugars. For example, the glucose isomer a-glucopyranose, upon dissolving in water, undergoes conversion into its [3] form until the equilibrium at approximately 37.5:62.5 in the respective mole fractions is reached [19]. The rate of reaction, determined by fitting a monoexponential model, is highly dependent on experimental conditions, primarily the temperature.

In high-field NMR spectroscopy, glucose isomers are typically quantified by integrating the peaks of the anomeric proton that occur at 5.2 and 4.6 ppm for α- and β-forms respectively (see FIG. 6). Unfortunately, these peaks are often obstructed by the much stronger peak of water at ≈4.75 ppm, which distorts the baseline and complicates integration. The use of deuterated solvent alleviates this problem, but notably slows down the reaction, which may be undesirable. Likewise, water suppression techniques inevitably introduce scaling into the acquired spectrum reducing the accuracy of quantification and thus should be avoided if possible. The spectrum resolution is further reduced at lower field strengths of benchtop NMR; separate anomeric peaks can no longer be clearly observed and integrated. Due to the difference of optical properties of the glucose isomers, their interconversion has been traditionally studied with polarimetric methods. However, the present NMR technique provides a convenient alternative.

To illustrate the suitabilitiy of the present technique, a 0.5M solution of glucose was prepared in 100% deionized H2O, and immediately placed into a sample tube, and transfered to the benchtop NMR instrument. A series of spectra was acquired in 45 s intervals over the course of several hours. The temperature inside the bore of the magnet was kept at approximately 30° C.

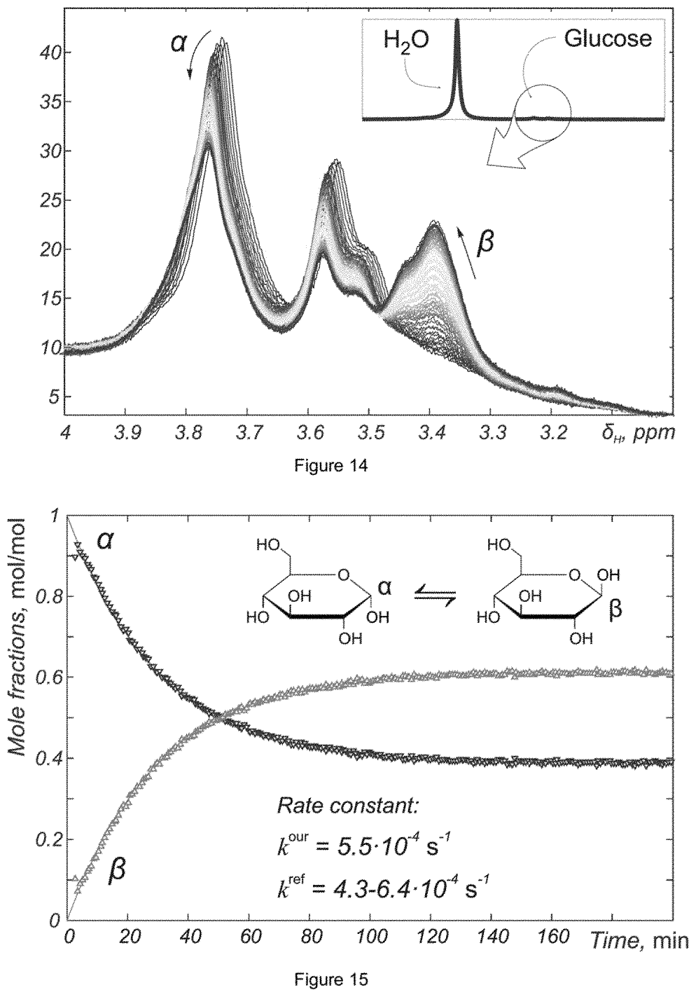

FIG. 14 shows an overlay of the acquired spectra. Relative concentrations of the glucose isomers were estimated using the present method for each spectral plane; FIG. 15 shoes these concentrations plotted with respect to the reaction time. The rate constant estimated as the parameter of the fitted monoexponential law was found to be 5.5×10⁻⁴ s⁻¹, which is within the range of reported reference values [19]. While the signal to noise ratio in this dataset is relatively high (the ratio of the height of glucose peaks to the standard deviation of noise is ≈10), the close proximity of the significantly more intense water peak notably distorts the baseline and complicates quantification. Despite these challenges, the present method achieves stable reconstruction with low deviation of estimates from the underlying exponential model of the experiment, using a benchtop NMR instrument.

Quantification of Controlled Mixtures

In most applications, the total amount of a specific sugar—but not each of its isomers separately—is of primary interest.

Mixtures of solid glucose, fructose, and sucrose were gravimetrically prepared with predefined mole fractions of each constituent and dissolved in 100% deionized H2O. About 4-5 g of each solid mixture was measured and water added to obtain 15 mL of the resulting solution (which, depending on the ratio of sugars, corresponds to molarity of ≈1 M). The manufacturer-specified purities of the components was over 99.0%, and the balance had an instrument accuracy of 0.1 mg (provided in the calibration protocol of the manufacturer).

Since the equilibrium concentrations of different isomers cⱼ are known (see Table 3), we construct a hierarchical model similar to the one shown in FIG. 16, and define a (group) reference model for each sugar as a sum of models of its isomers uⱼ included in fixed proportion u=Σⱼcⱼuⱼ. Similarly, we model two moieties of sucrose separately and then combine them in equal ratios. Thus, when processing complex mixtures, such as fruit juices, we effectively reduce the number of degrees of freedom, which facilitates model fitting, and directly quantify the total amount of each sugar. As shown in FIG. 13, average uncertainties in quantifying mixtures of the three sugars are expected to be comparable to the analysis of fructose isomers or the sucrose moieties.

The results of quantification are summarized in Table 4 and are compared to the relative concentrations estimated gravimetrically. The uncertainties in the gravimetric mole fractions are determined by means of error propagation based on the accuracy of laboratory balances and are expected to be no more than 10-4 mol/mol. The values estimated with the present method are equipped with respective 95% confidence intervals tighter than 0.01 mol/mol. We note that the maximum average deviation between the estimated and gravimetric values is in the order of 0.03 mol/mol. The error here arises primarily due to lineshape imperfections in the measured data and the resulting misfit of model signals. Specifically, accurate estimation of chemical shifts and J-coupling constants of fructose is notably challenging even with well-resolved high-field data due to significant overlaps of the spectra of its multiple isomers. Furthermore, closely located peaks of coupled protons—for example, H₆ₐ and H_{6b} in sucrose (see Table 2 above)—are extremely sensitive to deviations in their parameter values. These imperfect input models inevitably contribute to the final quantification error. Therefore, Equation 9 is regarded here as a lower bound on the confidence intervals of quantification. More comprehensive analysis of the contribution of uncertainty in specific model parameters to the final accuracy of estimation can be performed in a probabilistic setting.

TABLE 4
Results of model based quantification of five gravimetrically
prepared ternary mixtures of sugars obtained with a benchtop
instrument expressed in relative mole fractions, mol/mol.

| | | Glucose | Fructose | Sucrose | Avg. diff. |
|---|---|---|---|---|---|
| S1 | grav. | 0.332 | 0.332 | 0.336 | 0.012 |
| | estim. | 0.317 | 0.35 | 0.334 | |
| S2 | grav. | 0.168 | 0.666 | 0.166 | 0.019 |
| | estim. | 0.196 | 0.639 | 0.165 | |
| S3 | grav. | 0.666 | 0.168 | 0.166 | 0.031 |
| | estim. | 0.62 | 0.211 | 0.169 | |
| S4 | grav. | 0.168 | 0.168 | 0.664 | 0.012 |
| | estim. | 0.157 | 0.186 | 0.657 | |
| S5 | grav. | 0.417 | 0.417 | 0.167 | 0.008 |
| | estim. | 0.418 | 0.427 | 0.155 | |

Composition Analysis of Fruit Juices

Fruit juice samples were prepared fresh and filtered using a Millex syringe filter unit with 0.22 μm pore size to reduce turbidity. To avoid altering the natural composition of the samples, there was no pasteurization, or pH equilibration, and no deuterated water was added to the samples. The samples were measured using both a benchtop and a high-field NMR spectrometer.

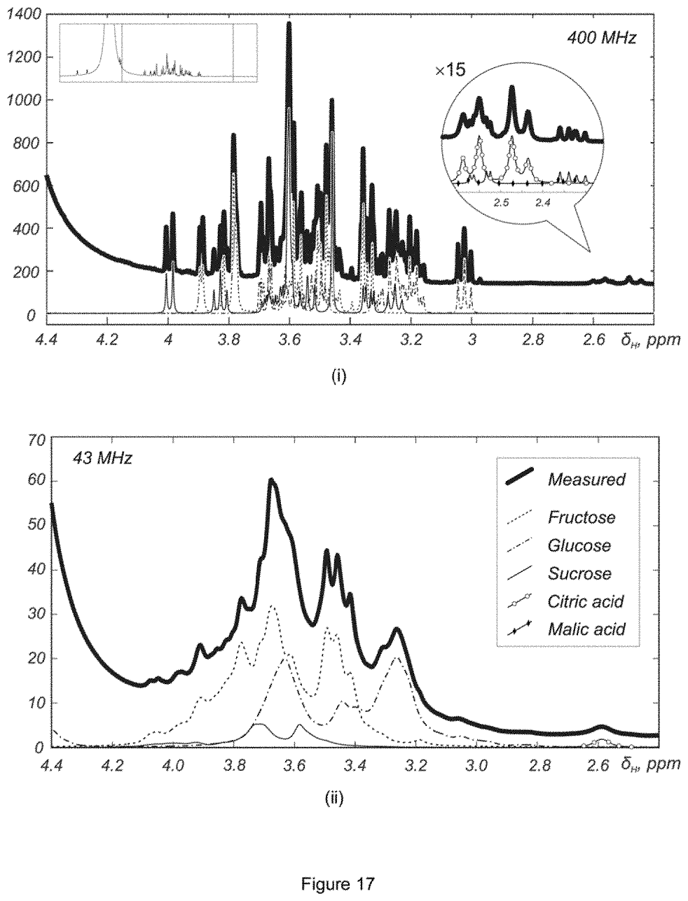

An example of acquired spectra on both instruments along with models fitted to them are shown in FIGS. 17(i) and 17(ii).

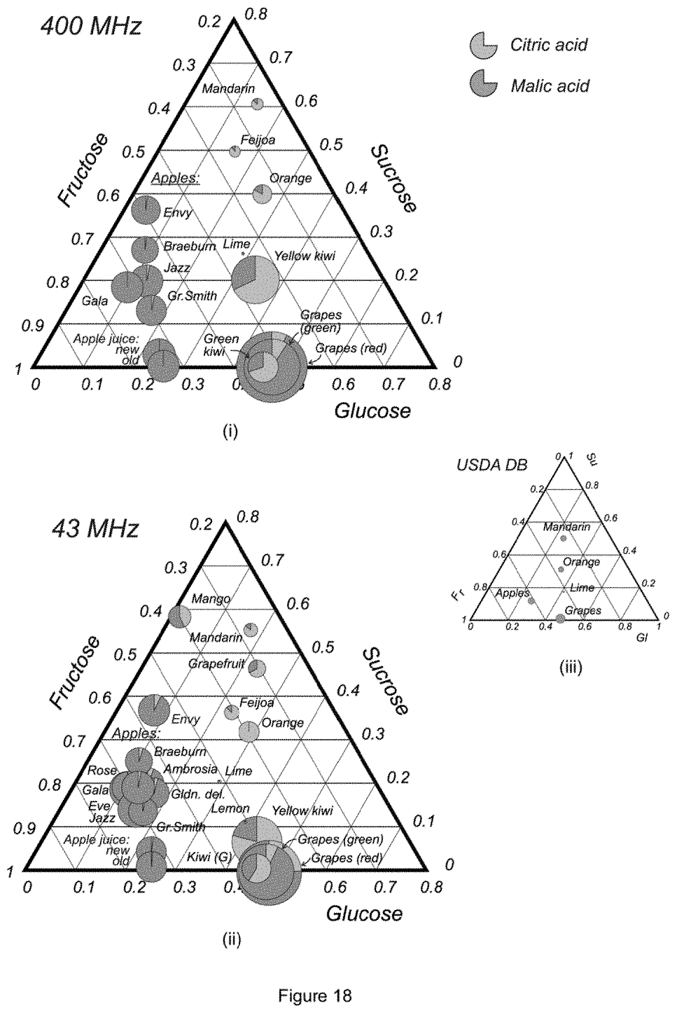

The amounts of glucose, sucrose, and fructose are estimated using the method described above. FIGS. 18(i) and (ii) show the relative mole fractions of the three sugars plotted on ternary diagrams. Each circle represents a separate sample; their radii are proportional to the total amounts of sugars estimated approximately with respect to the absolute intensity of the acquired signals. In addition to the three sugars, the ratio of predominant acids (malic and citric) are indicated using a pie chart within each plotted circle.

There is strong agreement between data from the high-field instrument (FIG. 18(i)) and data from the benchtop instrument FIG. 18(i). For example, differences among apples of several varieties are consistent in both datasets. Similarly, citrus fruit and grape juices occur in the corresponding regions of the diagrams with similar total amounts of sugars. While the spectra of sugars remain stable with varying pH, the chemical shifts of peaks of citric and malic acids deviate notably among multiple samples of juices. In addition to their typically lower concentration, this increases the resulting uncertainty. Nevertheless, the estimated mole fractions of the two predominant acids are, in most cases, consistent between the spectrometers.

A sample of bottled apple juice measured twice approximately six months apart (bottom left corner of the diagrams) shows the same trend towards decreasing of the amount of sucrose as the juice fermented notably. Similar changes occur with the yellow kiwifruit juice: this sample was measured on the high-field spectrometer immediately after preparation but two weeks later with the benchtop instrument. This indicates the potential application of portable benchtop instruments for accurate on-line monitoring of fermentation reactions, which are of great importance in the food industry, using the techniques described herein.

Finally, FIG. 18 shows a similar ternary diagram for composition of some fruit juices using data from the Nutrition Database of the US Department of Agriculture [20]. These reference values are in agreement with the findings of our analysis.

Analysis of Alcohol Mixture

Figure B15 is a further illustration of the application of the present quantitative analysis techniques, this time applied to a mixture of alcohols, namely ethanol and propanol.

Two samples with different relative concentrations of ethanol and propanol were prepared gravimetrically and analysed using the aforementioned benchtop spectrometer operating at 43 MHz frequency. The resulting spectra displayed significant overlap of the peaks of the two chemical species. This, along with the effects of the closely located strong peak of water, make this exemplary alcohol mixture challenging to analyse using conventional peak integration techniques with benchtop NMR data.

Due to the significant peak overlap of different species, using peak integration, one can only find aggregate integral values for groups of peaks at approximately 3.5 ppm and 0.5 to 2.0 ppm. Then, the sought molar fractions xEth and xpro can be recovered by taking into account different number of protons of ethanol and propanol contributing to each integral (see equations in the Figure). However, difficulties in achieving accurate baseline correction, specifically due to the influence of the water peak, contribute to poor performance of peak integration.

The present method overcomes this difficulty by using a separate model for the water peak to eliminate the baseline. Moreover, the quantum mechanical formulation of the chemical species accounts for higher order coupling effects most notably seen in the spectrum of propanol. This leads to much better agreement of the estimated mole fractions with the gravimetric values (see the tables in the top left corners of each plot).

Analysis of Thiamine and Pyridoxine Vitamin Mixture

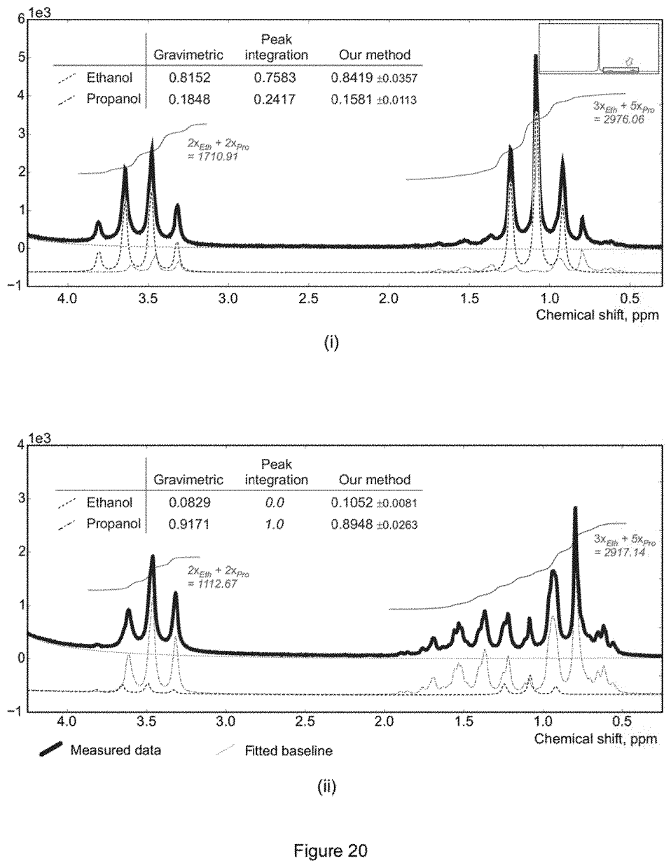

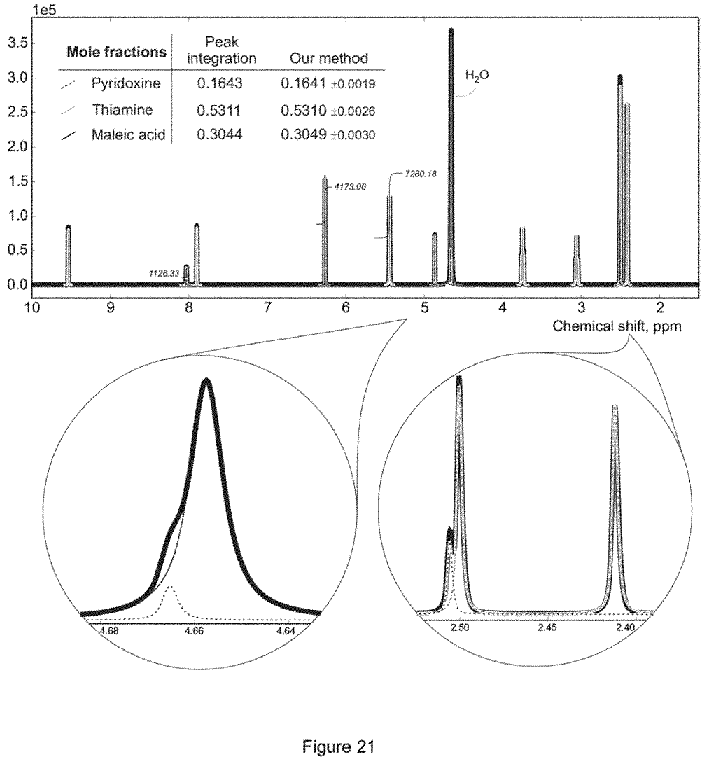

As a further example, FIG. 20 presents experimental results applying the present quantitative analysis techniques to a mixture of vitamins.

A sample containing vitamins B1 (thiamine), B6 (pyridoxine), and maleic acid in deuterated water is measured using the aforementioned high-field spectrometer operating at 400 MHz ¹H frequency. Models of each chemical specie and water are fitted using the above described methods. These models are shown in the various curves of in FIG. 20.

For comparison, the spectra for the mixture was analysed using peak integration Peaks chosen for integration are indicated in FIG. 20 with schematic integration curves and corresponding values of their areas. Peak integration is the method of choice for processing spectra such as this, with well resolved non-overlapping peaks, as it performs reliably after suitable phase and baseline correction is applied. In contrast, such examples are generally more challenging for model-based methods, since any line shape imperfections unaccounted for in the model can potentially impact the quantification.

The relative mole fractions estimated using the present method strongly agree with those of peak integration (see the Table in FIG. 20). Furthermore, the present method advantageously enables processing of the overlapping peaks shown in the enlargements of FIG. 20).

The experimental results presented above are restricted to simple one-pulse ¹H experiments and homonuclear couplings, augmenting the set of proton chemical shifts and J-couplings {δ^{H}, J^{HH}} with information about heteronuclear coupling, {δ^{C}, J^{HC}}, allows one to take into account ¹³C satellites in proton spectra in a principled way or even easily model higher dimensional experiments, such as HSQC, if desired.

Preferred embodiments of the invention have been described by way of example only and modifications may be made thereto without departing from the scope of the invention.

### REFERENCES

[1] M. Tiainen, P. Soininen and R. Laatikainen, “Quantitative Quantum Mechanical Spectral Analysis (qQMSA) of 1H NMR spectra of complex mixtures and biofluids,” Journal of Magnetic Resonance, vol. 242, pp. 67-78, 2014.
[2] I. Kuprov, N. Wagner-Rundell and P. J. Hore, “Polynomially scaling spin dynamics simulation algorithm based on adaptive state-space restriction,” Journal of Magnetic Resonance, vol. 189, pp. 241-250, 2007.
[3] A. M. Castillo, L. Patiny and J. Wist, “Fast and accurate algorithm for the simulation of NMR spectra of large spin systems,” Journal of Magnetic Resonance, vol. 209, pp. 123-130, 2011.
[4] J. Canavagh, W. J. Fairbrother, A. G. Palmer II, M. Rance and N. J. Skelton, “Theoretical Description of NMR Spectroscopy,” Protein NMR Spectroscopy 2nd Edit, 2007.
[5] S. A. Smith, W. E. Palke and J. T. Gerig, “The Hamiltonians of NMR. part I,” Concepts in Magnetic Resonance, vol. 4, pp. 107-144, 4 1992.
[6] H. J. Hogben, M. Krzystyniak, G. T. P. Charnock, P. J. Hore and I. Kuprov, “Spinach—A software library for simulation of spin dynamics in large spin systems,” Journal of Magnetic Resonance, vol. 208, pp. 179-194, 2011.
[7] L. J. Edwards and I. Kuprov, “Parallel density matrix propagation in spin dynamics simulations,” Journal of Chemical Physics, vol. 136, 2012.
[8] Y. Matviychuk, E. Harbou and D. J. Holland, “An experimental validation of a Bayesian model for quantification in NMR spectroscopy,” Journal of Magnetic Resonance, vol. 285, pp. 86-100, 12 2017.
[9] M. Bydder, “Solution of a complex least squares problem with constrained phase,” Linear Algebra and its Applications, vol. 433, pp. 1719-1721, 2010.
[10] I. Markovsky, “On the Complex Least Squares Problem with Constrained Phase,” SIAM Journal on Matrix Analysis and Applications, vol. 32, pp. 987-992, 2011.
[11] G. L. Bretthorst, Bayesian spectrum analysis and parameter estimation, Springer-Verlag, 1988.
[12] C. W. P. d. S. Grandizoli, F. R. Campos, F. Simonelli and A. Barison, “Grape juice quality control by means of 1H NMR spectroscopy and chemometric analyses,” Quimica Nova, vol. 37, pp. 1227-1232, 2014.
[13] N. Ogrinc, I. J. Kos̆ir, J. E. Spangenberg and J. Kidrc̆, “The application of NMR and MS methods for detection of adulteration of wine, fruit juices, and olive oil. A review,” Analytical and Bioanalytical Chemistry, vol. 376, pp. 424-430, 2003.
[14] M. U. Roslund, P. Tähtinen, M. Niemitz and R. Sjoholm, “Complete assignments of the 1H and 13C chemical shifts and JH, H coupling constants in NMR spectra of d-glucopyranose and all d-glucopyranosyl-d-glucopyranosides,” Carbohydrate Research, vol. 343, pp. 101-112, 2008.
[15] T. Barclay, M. Ginic-Markovic, M. R. Johnston, P. Cooper and N. Petrovsky, “Observation of the keto tautomer of D-fructose in D(2)O using (1)H NMR spectroscopy,” Carbohydrate Research, vol. 347, pp. 136-141, 2012.
[16] A. De Bruyn and J. Van Loo, “The identification by 1H- and 13C-n.m.r. spectroscopy of sucrose, 1-kestose, and neokestose in mixtures present in plant extracts,” Carbohydrate Research, vol. 211, pp. 131-136, 1991.
[17] A. De Bruyn, M. Anteunls and G. Vereegge, “A 1H-n.m.r. study of D-fructose in D2O.,” vol. 41, pp. 295-297, 1975.
[18] M. Jaseja, A. S. Perlin and P. Dais, “Two-dimensional NMR spectral study of the tautomeric equilibria of D-fructose and related compounds,” Magnetic Resonance in Chemistry, vol. 28, pp. 283-289, 1990.
[19] M. Nelson and F. M. Beegle, “Mutarotation of Glucose and Fructose,” J. Am. Chem. Soc., pp. 559-575, 1919.
[20] United States Department of Agriculture, Agricultural Research Service, USDA Food Composition Databases.
[21] M. Tiainen, Quantitative Quantum Mechanical Analysis of 1 H NMR Spectra.

1. A method of determining the concentrations of constituent chemical species in a mixture, comprising:
using nuclear magnetic resonance spectroscopy, acquiring an NMR measurement for a sample of the mixture;
for each of the constituent chemical species, retrieving a reference model representative of the NMR FID signal or frequency domain spectra from a database, each model having a number of parameters; wherein for at least one of the constituent chemical species, the model is a quantum mechanical model;
using a computer, generating a model signal for the mixture and adjusting some or all of the model parameters to fit the model signal to the measured data; and
based on the fitted model signal, calculating and displaying the concentrations of the constituent species in the sample.

2. A method as claimed in claim 1, wherein the acquired NMR measurement is obtained using a benchtop-type NMR spectrometer.

3. A method as claimed in claim 1 or 2, wherein the acquired NMR measurement is obtained using an NMR spectrometer of a type having a permanent magnet.

4. A method as claimed in claim 1, wherein the acquired NMR measurement is obtained using an NMR spectrometer with an operating frequency of less than 100 MHz.

5. A method as claimed in any preceding claim, further comprising the step of hierarchically arranging the reference models for the chemical species.

6. A method as claimed in claim 5, wherein the step of hierarchically arranging the reference models comprises forming one or more groups containing multiple constituent species, wherein constituent species within the same group display similar responses to specific experimental conditions; and assigning one or more model parameters at the group level to reduce the overall number of parameters.

7. A method as claimed in any preceding claim, wherein at least one of the constituent chemical species reference models is specified in terms of the transition peaks with parameters found by diagonalization of the spin Hamiltonian.

8. A method as claimed in any preceding claim, wherein at least one of the constituent chemical species reference models is a quantum mechanical model utilising temporal propagation.

9. A method as claimed in any preceding claim, wherein different types of models are used to generate the reference signals of different constituent species, the models each being selected from: a base model of spectra peaks, a quantum mechanical model utilising diagonalization, a quantum mechanical model utilising temporal propagation, and experimental data.

10. A method as claimed in any preceding claim, wherein the reference signal for at least one of the chemical species is independent of the NMR instrument's field strength.

11. A method as claimed in any preceding claim, wherein the mixture contains K chemical species, and the NMR signal (x) for the mixture is a superposition of the reference signals uₖ of the constituent chemical species, modelled according to:
$$x = {e}^{i {ϕ}_{0}} {\sum }_{k = 1}^{K} {c}_{k} {u}_{k} ( {θ}_{k} , τ ) ,$$
where: θₖ represents the model parameters, τ is the ring-down delay, φ₀ is the global phase shift and cₖ are intensity estimators that are proportional to the concentration of the corresponding species k.

12. A method as claimed in claim 11, wherein the model parameters comprise one or more of: chemical shifts of the peaks, relaxation rates, peak intensities, and J-coupling constants.

13. A method as claimed in any preceding claim, wherein the acquired NMR measurement is obtained using single pulse ¹H NMR

14. A method as claimed in any preceding claim, the method further comprises the step of using marginal posterior distributions of the intensity estimators to analyse the uncertainties in their calculated values.

15. A method as claimed in claim 14, further comprising the step of using an MCMC algorithm to sample the posterior distribution of the fitted model to estimate the uncertainty of the model parameters.

16. A method as claimed in claim 14 or 15, wherein, along with the step of displaying the concentrations of the constituent species in the sample, the confidence or credible intervals relating to the concentrations are displayed.

17. A method as claimed in claim 16 wherein the confidence or credible intervals are calculated using a robust variance estimator taking into account the residual signal between the model signal and the NMR measurements.

18. A method as claimed in any preceding claim, wherein the step of fitting the model signal, or calculating the concentrations of the constituent species further comprises the step of performing line shape correction.

19. A method as claimed in any preceding claim, wherein the step of fitting the model signal, or calculating the concentrations of the constituent species comprises utilising a generalized least squares (GLS) estimator.

20. A method as claimed in claim 19, wherein the generalized least squares (GLS) estimator treats possible model misspecification as additional non-isotropic noise.

21. A method as claimed in claim 19 or 20, wherein, in the frequency domain, the variance of the noise assumed to be proportional to the absolute value of the derivative of the modelled NMR spectra.

22. A method as claimed in any preceding claim, wherein the mixture comprises sugars.

23. A method as claimed in claim 22, wherein the chemical species comprise one or more of glucose, fructose and sucrose.

24. A method as claimed in claim 22 or 23, wherein the mixture is a fruit juice.

25. A method as claimed in any one of claims 1 to 21, wherein the chemical species comprise one or more alcohols.

26. A system for determining the concentrations of constituent chemical species in a mixture, comprising:
a nuclear magnetic resonance (NMR) spectrometer, for acquiring an NMR measurement of a sample of the mixture;
a computer storage media comprising a database of NMR FID signal or frequency domain spectra reference models for each constituent species, each model having a number of parameters; wherein for at least one of the constituent chemical species, the model is a quantum mechanical model;
a model signal generator configured to generate a model signal for the mixture and adjust some or all of the model parameters to fit the model signal to the measured data, and thereby calculate the concentrations of each of the constituent species in the sample; and
a user interface for receiving input commands from a user and for displaying the calculated concentrations of each of the constituent species.

27. A system as claimed in claim 26, wherein the model generator comprises means for combining the reference models for each constituent species to generate the model signal.

28. A system as claimed in claim 26 or 27, wherein the NMR spectrometer is a benchtop-type spectrometer.

29. A system as claimed in any one of claims 26 to 28, wherein the NMR spectrometer of a type having a permanent magnet.

30. A system as claimed in any one of claims 26 to 29, wherein the NMR spectrometer operates at less than 100 MHz.

31. A system as claimed in any one of claims 26 to 30, comprising means for hierarchically arranging the chemical species and their respective reference models.

32. A system as claimed in any one of claims 26 to 31, wherein at least one of the constituent chemical species reference models in the database is specified in terms of the transition peaks with parameters found by diagonalization of the spin Hamiltonian.

33. A method as claimed in any one of claims 26 to 32, wherein at least one of the constituent chemical species reference models in the database is a quantum mechanical model utilising temporal propagation.

34. A system as claimed in any one of claims 26 to 33, wherein the database comprises different types of reference models for different constituent species, the model types being selected from: a base model of spectra peaks, a quantum mechanical model utilising diagonalization, a quantum mechanical model utilising temporal propagation, and experimental data.

35. A system as claimed in any one of claims 26 to 34, wherein at least one of the reference models in the database is independent of the field strength of the NMR spectrometer.

36. A system as claimed in any one of claims 26 to 35, wherein the model signal generator combines the reference signals uₖ for a mixture containing K chemical species using superposition, and generates the NMR signal (x) for the mixture according to:
$$x = {e}^{i {ϕ}_{0}} {\sum }_{k = 1}^{K} {c}_{k} {u}_{k} ( {θ}_{k} , τ ) ,$$
where: θₖ represents the model parameters, τ is the ringdown delay, φ₀ is the global phase shift and cₖ are intensity estimators that are proportional to the concentration of the corresponding species k.

37. A system as claimed in claim 36, wherein the model parameters comprise one or more of: chemical shifts of the peaks, relaxation rates, peak intensities, and J-coupling constants.

38. A system as claimed in any one of claims 26 to 37, further comprising the step of using an MCMC algorithm to sample to posterior distribution of the fitted model to estimate the uncertainty of the model parameters.

39. A system as claimed in any one of claims 26 to 38, along with the step of displaying the concentrations of the constituent species in the sample, the confidence intervals relating to the concentrations are displayed.

40. A non-transient computer readable medium containing program instructions for causing a computer to:
upon receiving an NMR measurement for a sample of a mixture of a number of known constituent chemical species, retrieve a reference model representative of the NMR FID signal or frequency domain spectra for each specie from a database, each model having a number of parameters; at least one of the retrieved reference models being a quantum mechanical model;
generate a computer readable model signal for the mixture and adjust some or all of the model parameters to fit the model signal to the measured data; and based on the fitted model signal, calculate and display the concentrations of the constituent species in the sample.
